(19) United States
(12) Patent Application Publication
(10) Pub. No.: US 2026/0027230 A1
(43) Pub. Date: Jan. 29, 2026

(54) DRUG DELIVERY SYSTEM, TREATMENT KIT, AND METHOD FOR INHIBITING PROLIFERATION OR METASTASIS OF CANCER CELL

(71) Applicant: China Medical University, Taichung City (TW)

(72) Inventors: Wen-Lung Ma, Taichung City (TW); Wei-Chung Cheng, Taichung City (TW); Juan-Cheng Yang, Tainan City (TW)

(73) Assignee: China Medical University, Taichung City (TW)

(21) Appl. No.: 19/283,536

(22) Filed: Jul. 29, 2025

Related U.S. Application Data
(60) Provisional application No. 63/676,450, filed on Jul. 29, 2024.

(51) Int. Cl. A61K 47/69; A61K 31/015; A61K 31/192; A61K 31/197; A61K 31/203; A61K 45/06; A61K 47/64; A61P 35/04

(52) U.S. Cl. CPC A61K 47/6917; A61K 31/015; A61K 31/192; A61K 31/197; A61K 31/203; A61K 45/06; A61K 47/64; A61P 35/04

(57) ABSTRACT

A drug delivery system, a treatment kit, and a method for inhibiting a proliferation or a metastasis of a cancer cell are provided. The drug delivery system includes a very low density lipoprotein carrier, a target ligand and a pharmaceutically active ingredient. The target ligand is conjugated to the very low density lipoprotein carrier, and the target ligand has a binding specificity to a very low density lipoprotein receptor. The pharmaceutically active ingredient is encapsulated in the very low density lipoprotein carrier.

### RELATED APPLICATIONS

This application claims priority to U.S. Provisional Application Ser. No. 63/676,450, filed Jul. 29, 2024, which is herein incorporated by reference.

### SEQUENCE LISTING XML

The Sequence Listing associated with this application is filed in electronic format via Patent Center and is hereby incorporated by reference into the specification in its entirety. The name of the text file containing the Sequence Listing is CP-7212-US_SEQ_LIST_202510.xml. The size of the text file is 4,773 bytes, and the text file was created on Oct. 9, 2025.

### BACKGROUND

### Technical Field

The present disclosure relates to drug delivery system, a treatment kit and a method for inhibiting a proliferation or a metastasis of a cancer cell. More particularly, the present disclosure relates to a lipoprotein-based drug delivery system, a treatment kit and a method for inhibiting a proliferation or a metastasis of a cancer cell.

### Description of Related Art

Cancer represents a heterogeneous group of diseases characterized by uncontrolled cellular proliferation with the potential for invasion or metastasis to distant tissues. This distinguishes them from benign tumors, which lack metastatic potential. The clinical presentation of cancer exhibits significant variability, but may include the formation of a palpable mass, atypical bleeding, persistent cough, unexplained weight loss, and alterations in bowel habits. Over one hundred distinct types of cancer have been identified in humans, making it the second leading cause of mortality worldwide.

Surgery, chemotherapy, and irradiation are the mainstream therapeutic approaches for cancer, chemotherapy being a systemic treatment and being an important component of treatment for cancer patients. However, cancer treatments can cause side effects. For example, side effects are problems that occur when treatment affects healthy tissues or organs, leading to anemia, appetite loss, bleeding, constipation, delirium, diarrhea, edema, or fatigue. As a result, some treatments show the low specificity and off-target effects. Although there are some delivery systems developed, they still have many problems to be improved. Therefore, the development of the delivery system to improve the specificity, penetration and efficacy of treatment is very urgently needed.

Thus, there is an unmet need in the art for developing a nanoparticle as a multifunctional delivery nanoparticle for carrying pharmaceutically active substances. Furthermore, there is an urgent need in this art for constructing a method capable of treating cancer by solving the above problems in the field and meet the clinical needs.

### SUMMARY

According to one aspect of the present disclosure is to provide a drug delivery system. The drug delivery system includes a very low density lipoprotein carrier, a target ligand and a pharmaceutically active ingredient. The target ligand is conjugated to the very low density lipoprotein carrier, and the target ligand has a binding specificity to a very low density lipoprotein receptor. The pharmaceutically active ingredient is encapsulated in the very low density lipoprotein carrier.

According to another aspect of the present disclosure is to provide a treatment kit. The treatment kit includes the aforementioned drug delivery system and a pharmaceutically acceptable carrier.

According to still another aspect of the present disclosure is to provide a method for inhibiting a proliferation or a metastasis of a cancer cell. The method includes administering the aforementioned treatment kit to a subject in need for a treatment of a cancer.

### BRIEF DESCRIPTION OF THE DRAWINGS

The present disclosure can be more fully understood by reading the following detailed description of the embodiment, with reference made to the accompanying drawings as follows:

FIG. 1A is a schematic diagram showing that patients from the CMUH-HCC cohort were subjected to the lipidomic analysis, transcriptomic analysis, and metabolomic analysis.

FIG. 1B is a Venn diagram plot for analysis of the CMUH-HCC cohort for lipidomic analysis, transcriptomic analysis, and metabolomic analysis.

FIG. 1C shows the ranking of association levels of lipid classes to hazard ratios (HR) in the CMUH-HCC cohort patients.

FIG. 1D shows the double bond number and carbon length of acyl chain of cholesterol-ester (CE) association to HR in the CMUH-HCC cohort patients.

FIG. 1E shows the Double bond number and carbon length of acyl chain of ether-linked lyso-phosphatidyl-ethanolamine (LPE O—) association to HR in the CMUH-HCC cohort patients.

FIG. 1F shows the double bond number and carbon length of acyl chain of ether-linked phosphatidyl-choline (PC O—) association to HR in the CMUH-HCC cohort patients.

FIG. 1G shows the double bond number and carbon length of acyl chain of ether-linked phosphatidyl-ethanolamine (PE O—) association to HR in the CMUH-HCC cohort patients.

FIG. 2A shows that the lipidomic dataset of the CMUH-HCC cohort were divided into two groups by using unsupervised lipidomic cluster analysis executed through the t-SNE dimension reduction analysis.

FIG. 2B shows analysis results of overall survival probability comparing the t-SNE_A group and t-SNE_B group.

FIG. 2C shows the differential lipid analysis comparing t-SNE_A patients to t-SNE_B patients.

FIG. 2D shows the comparison of t-SNE_A and t-SNE_B groups according to the results of enrichment analyses conducted on the identified lipid class.

FIG. 2E and FIG. 2F show analysis results of migration activity and invasion activity in the Tong cells and the Huh7 cells treated with PC O—, respectively.

FIG. 2G and FIG. 2H show analysis results of migration activity and invasion activity in the Tong cells and the Huh7 cells treated with PE O—, respectively.

FIG. 21 shows that the transcriptomic dataset of the CMUH-HCC cohort were divided into two groups by using unsupervised transcriptomic cluster analysis executed through the t-SNE dimension reduction analysis.

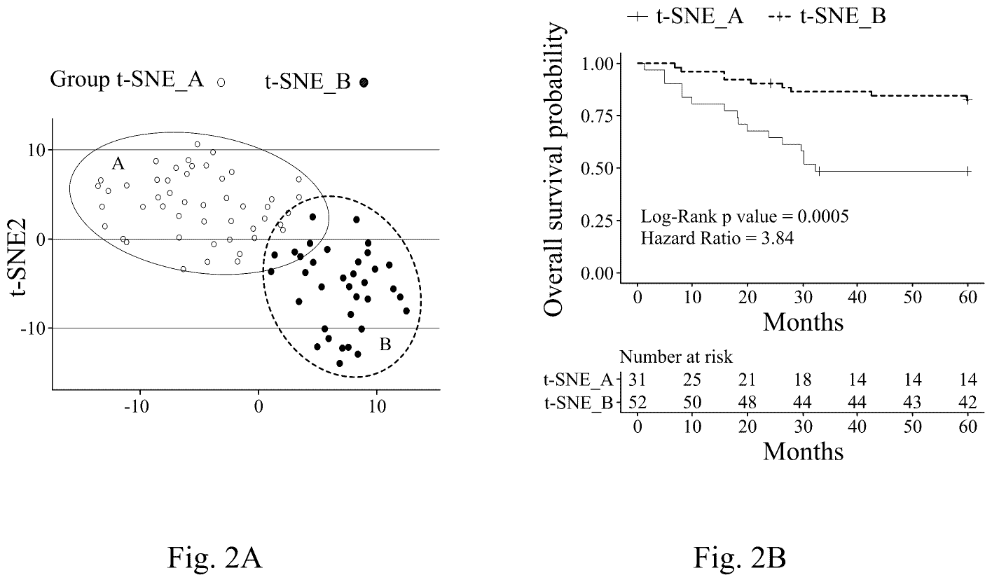

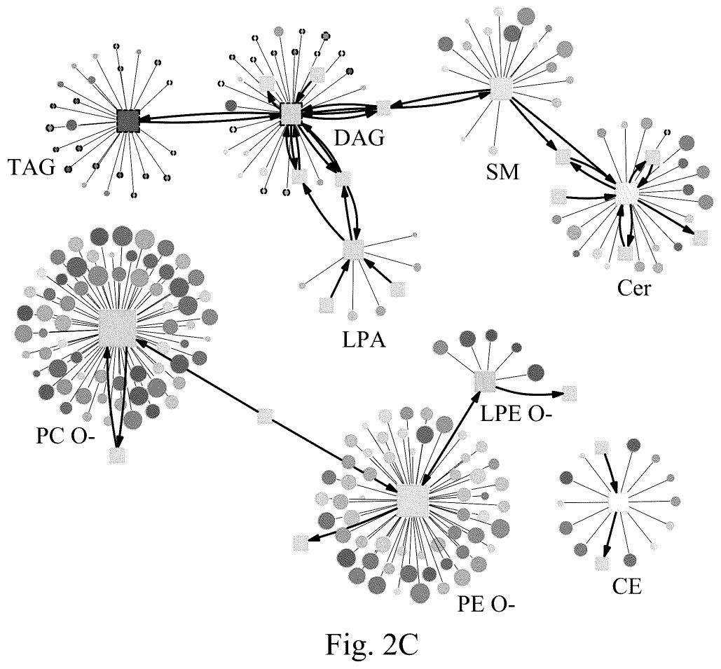

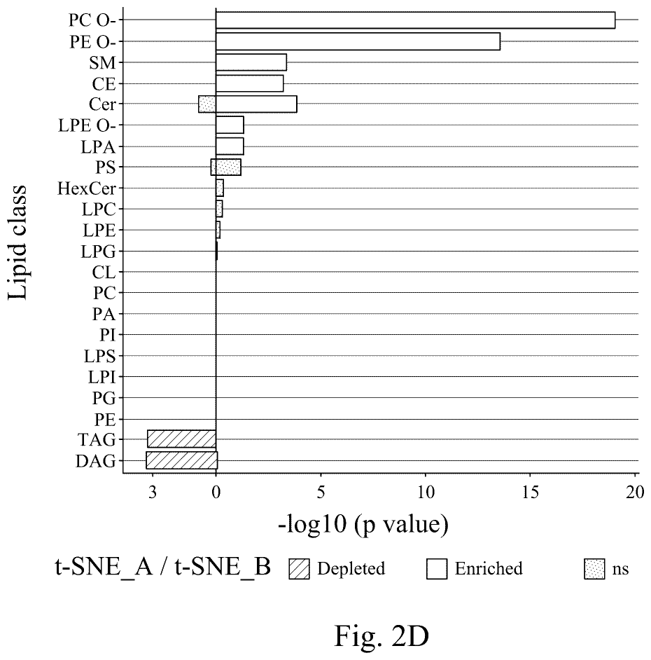

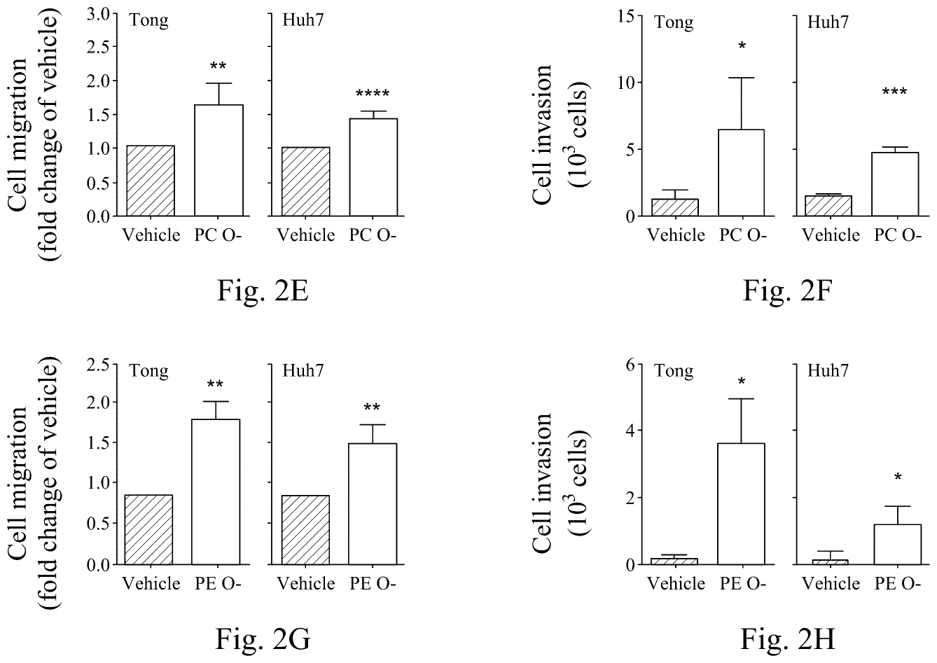

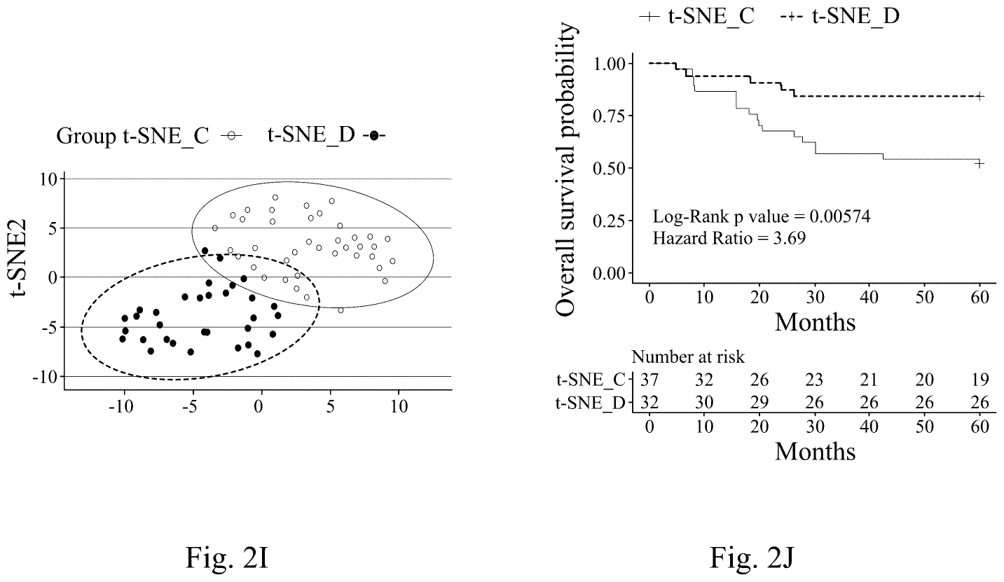

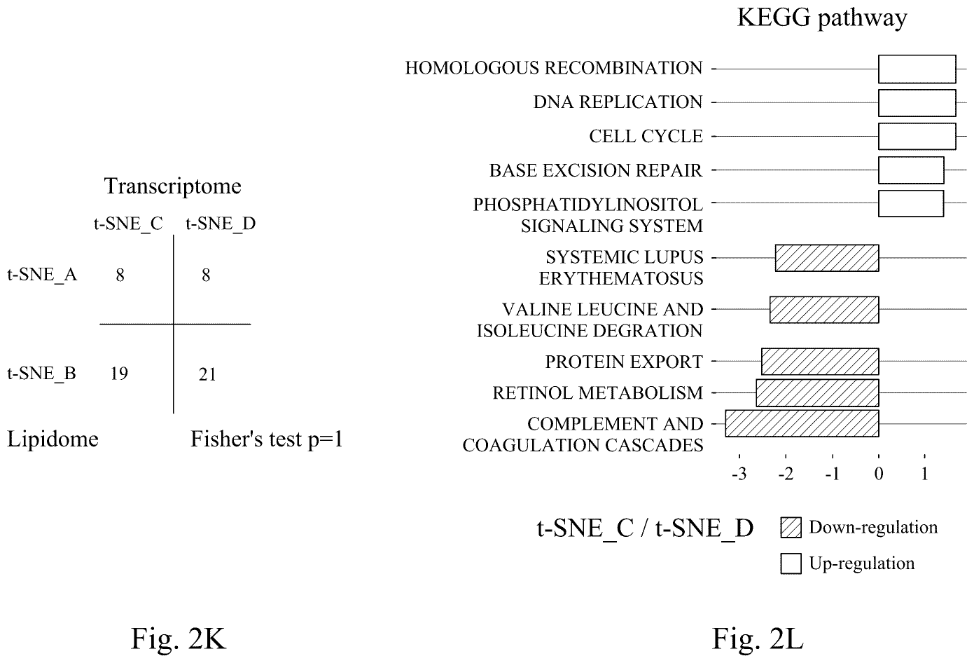

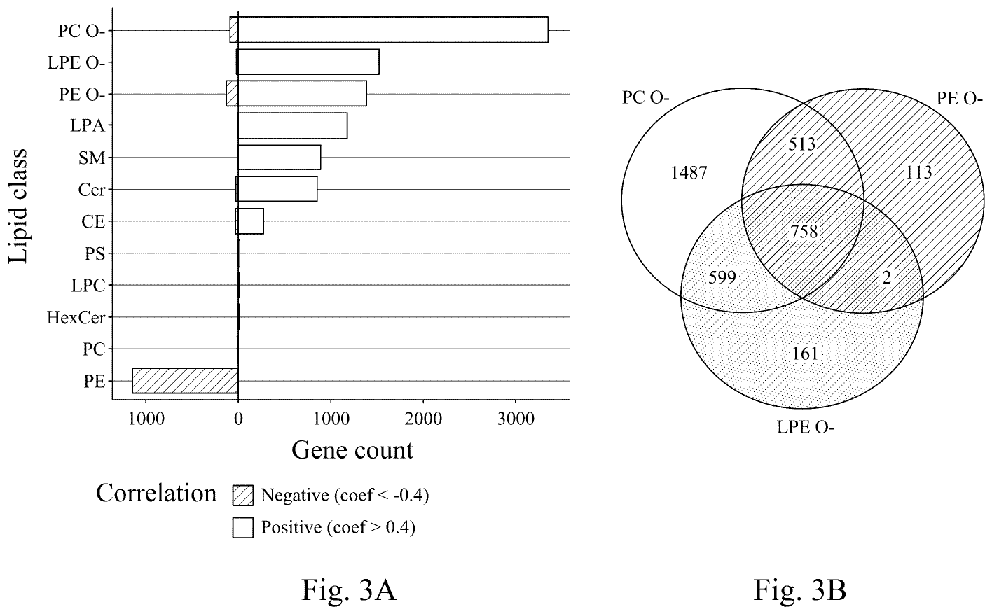

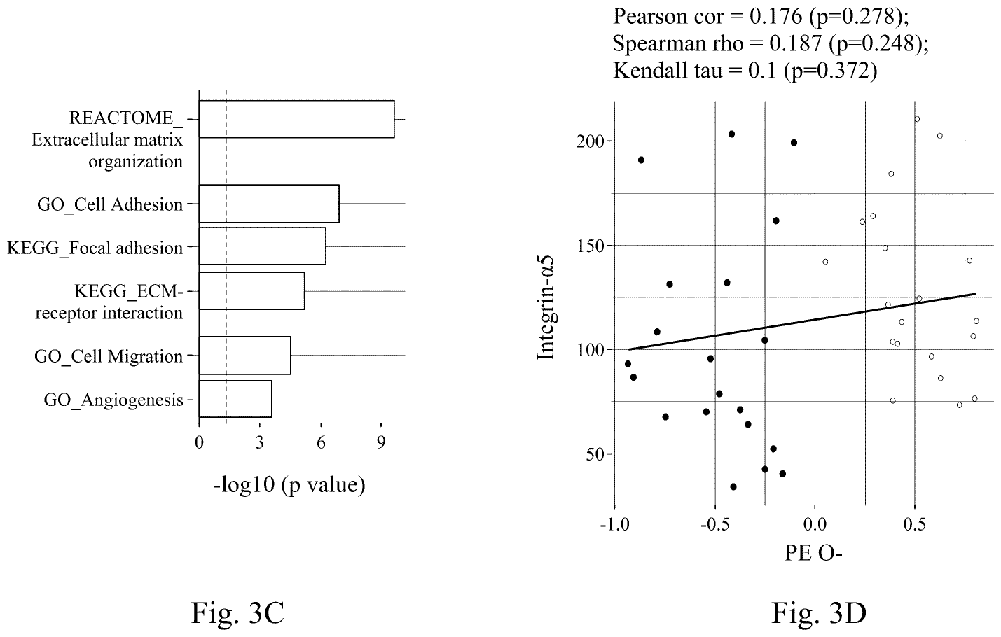

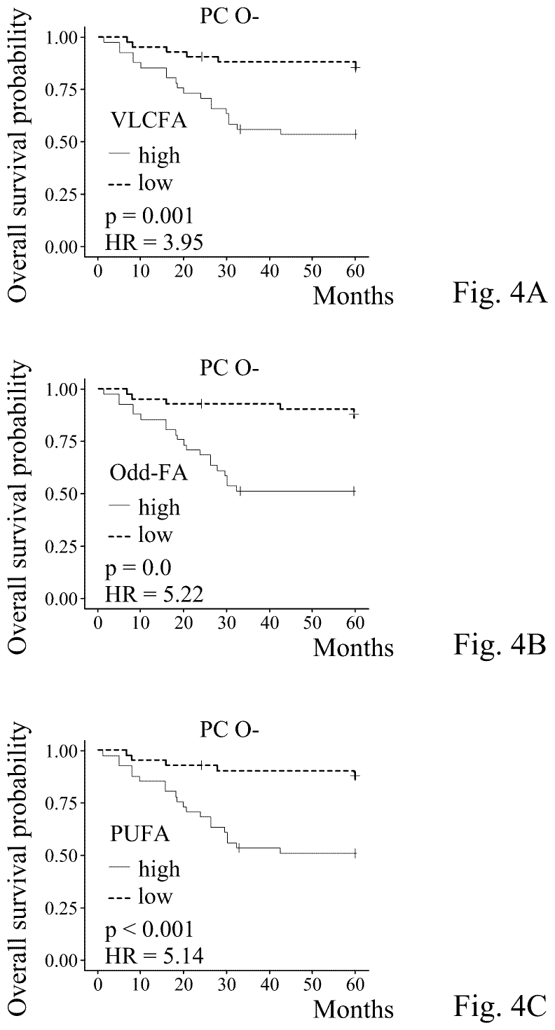

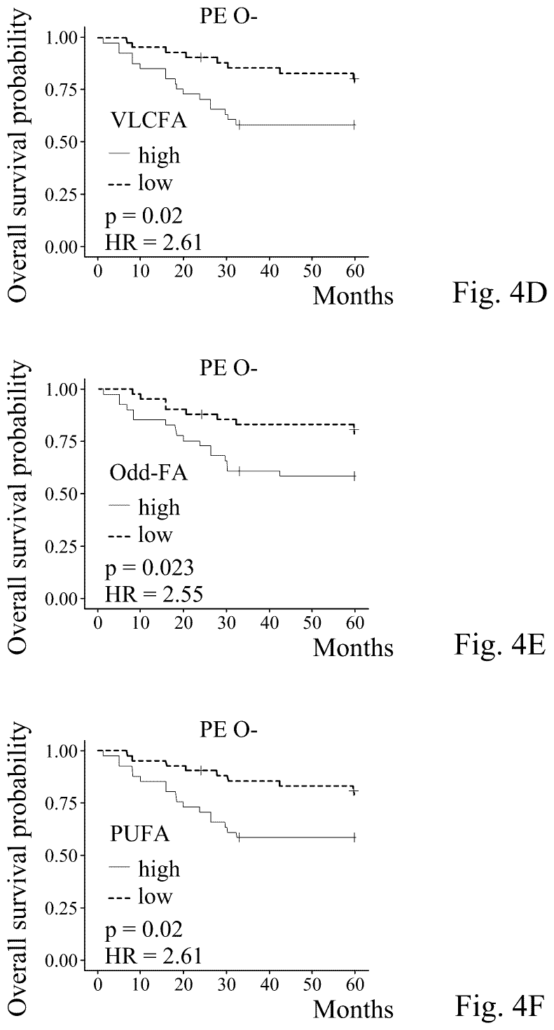

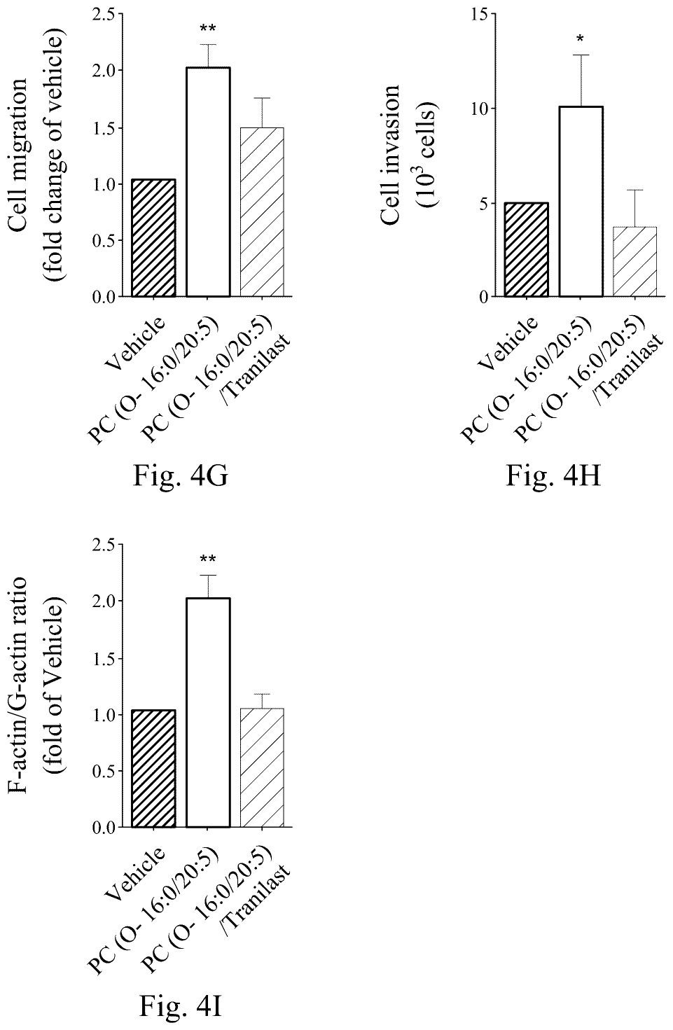

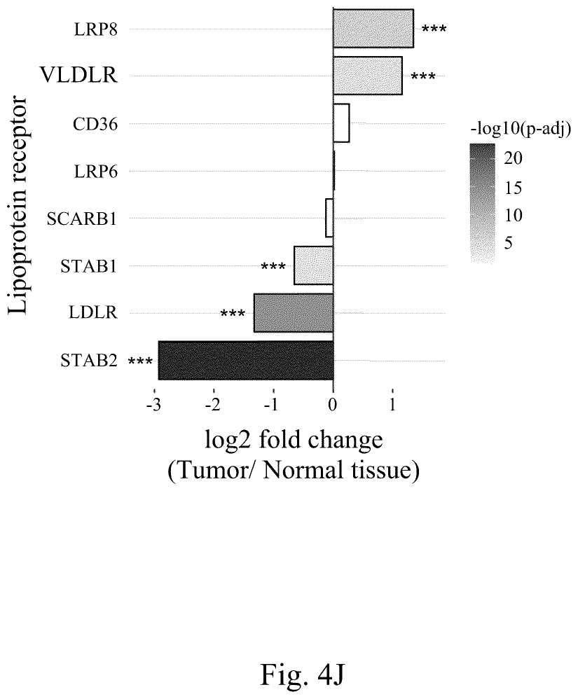

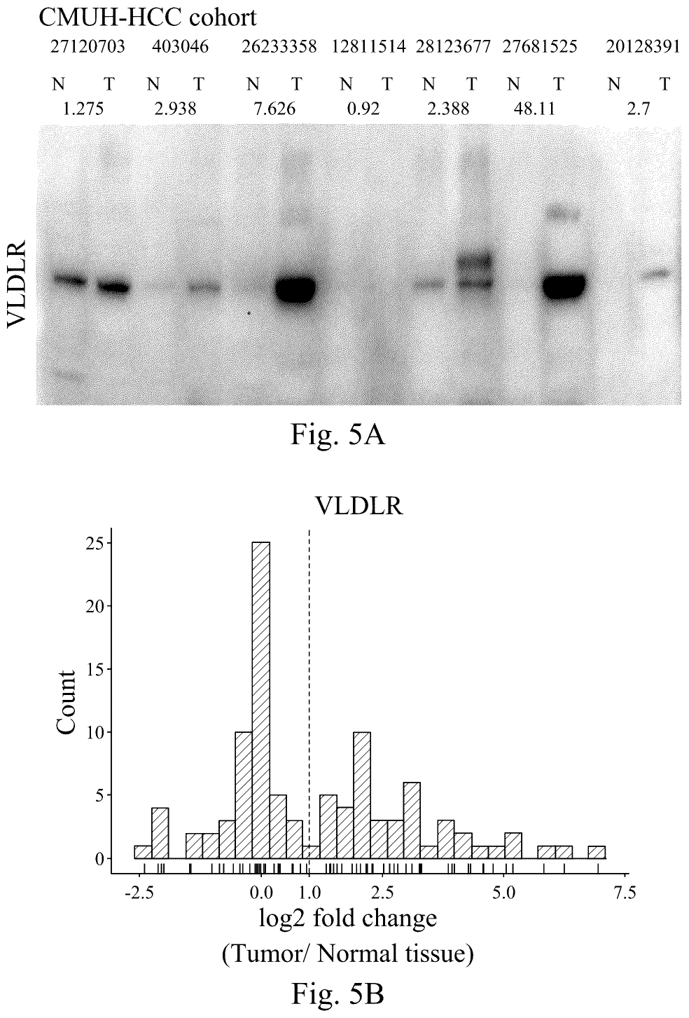

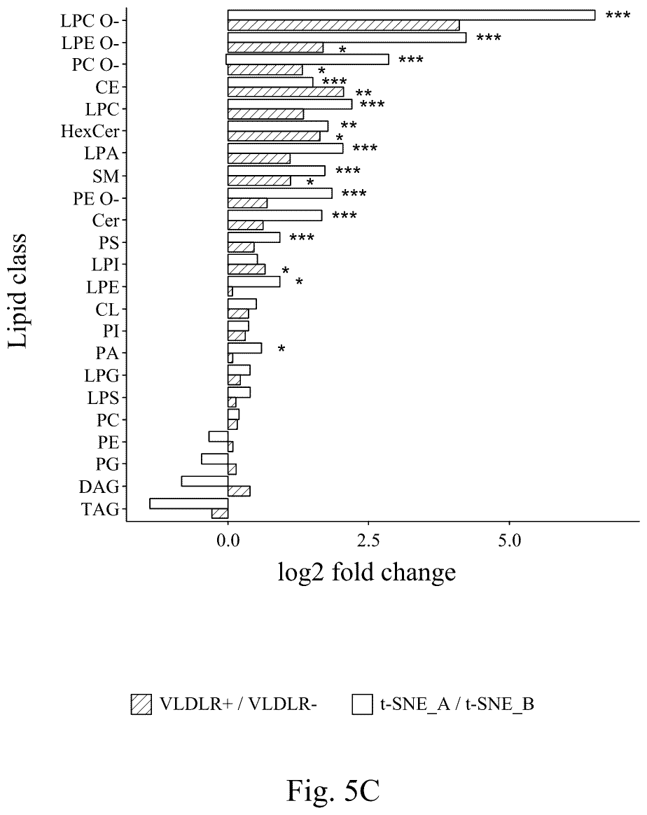

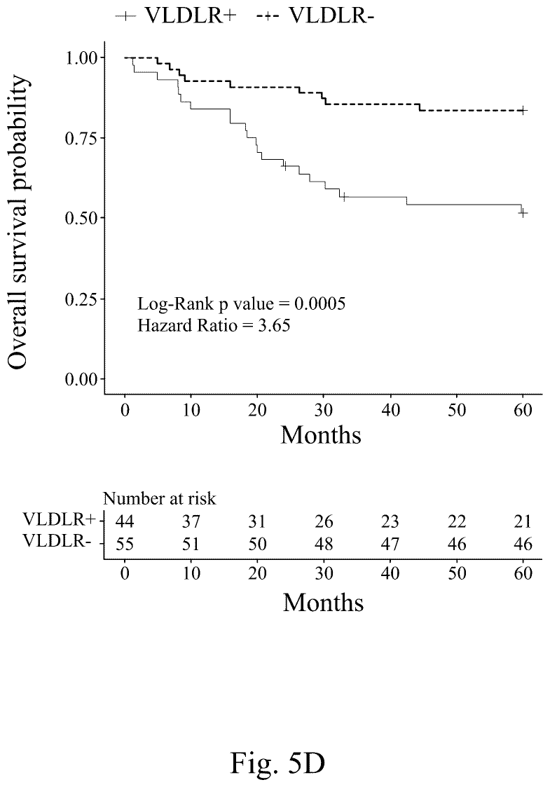

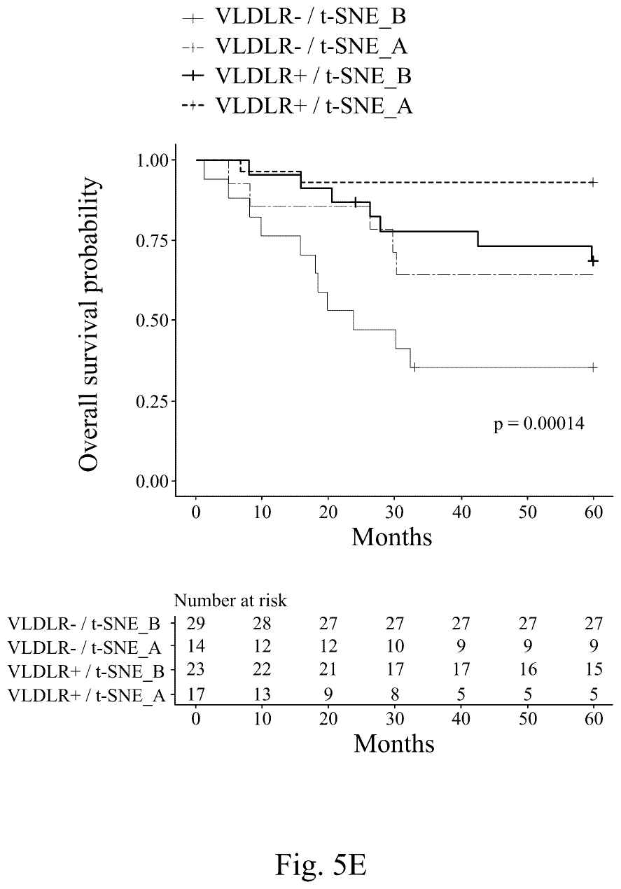

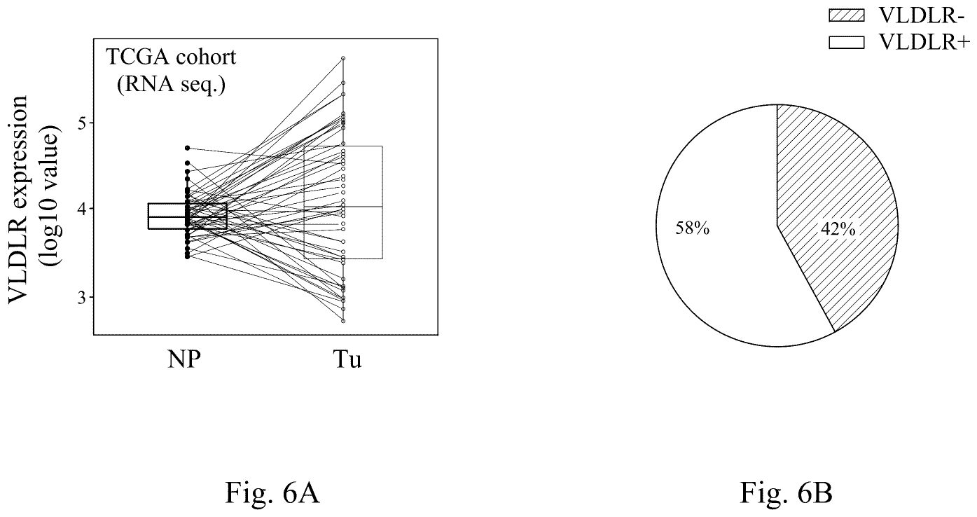

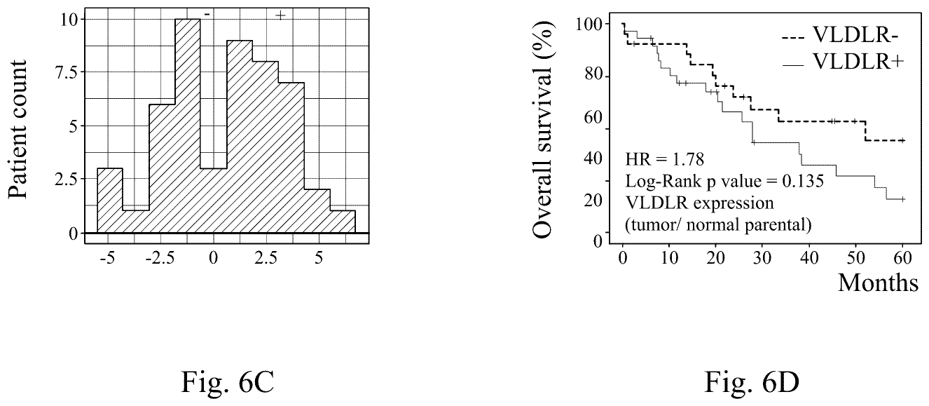

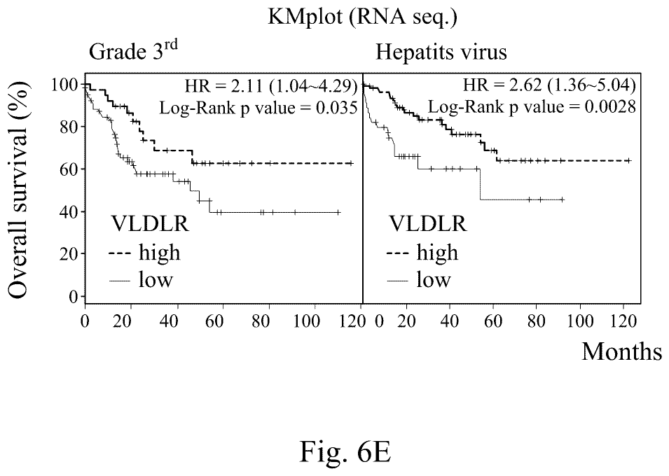

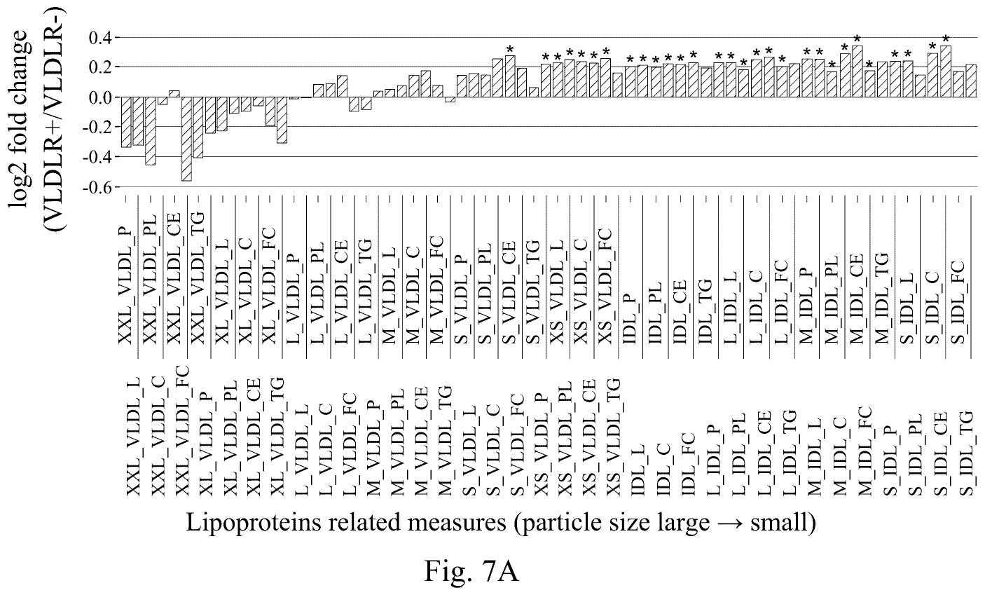

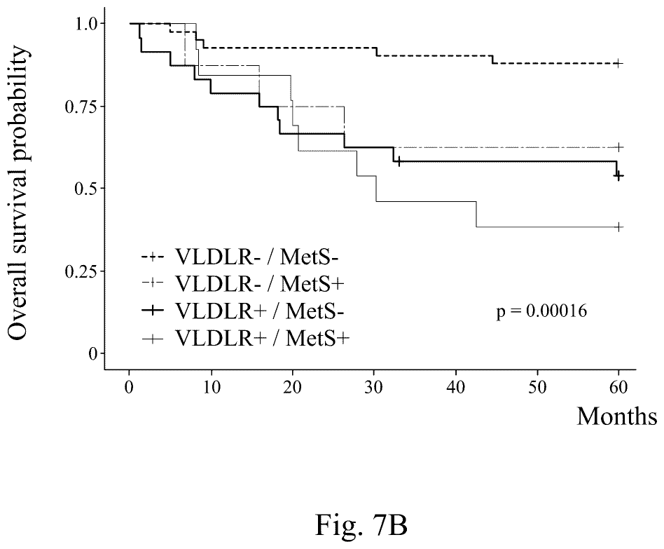

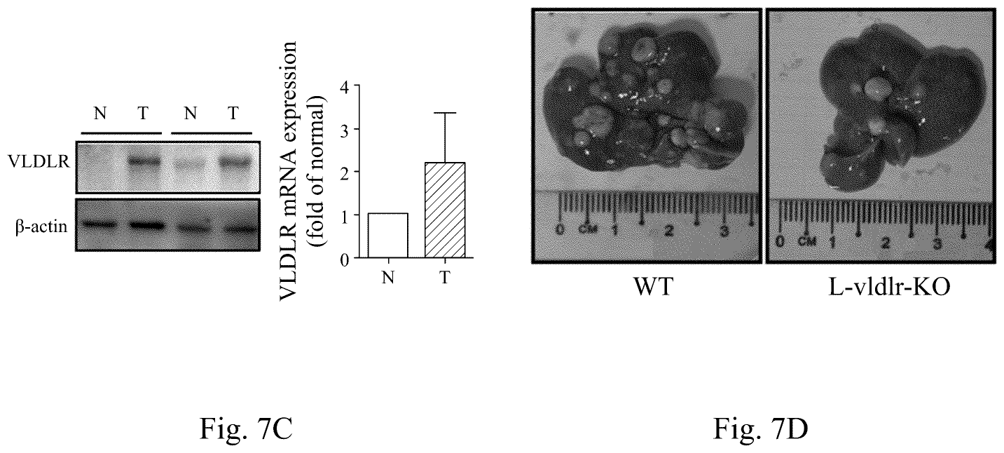

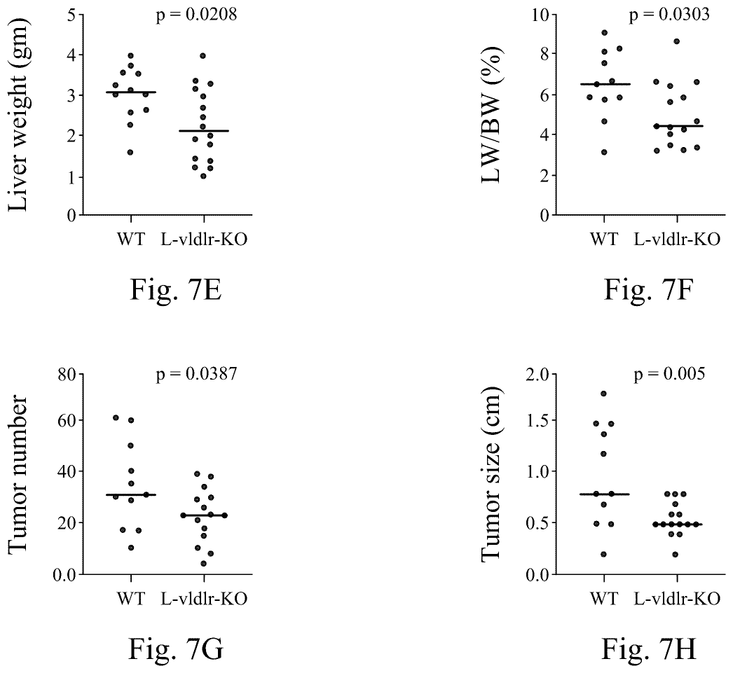

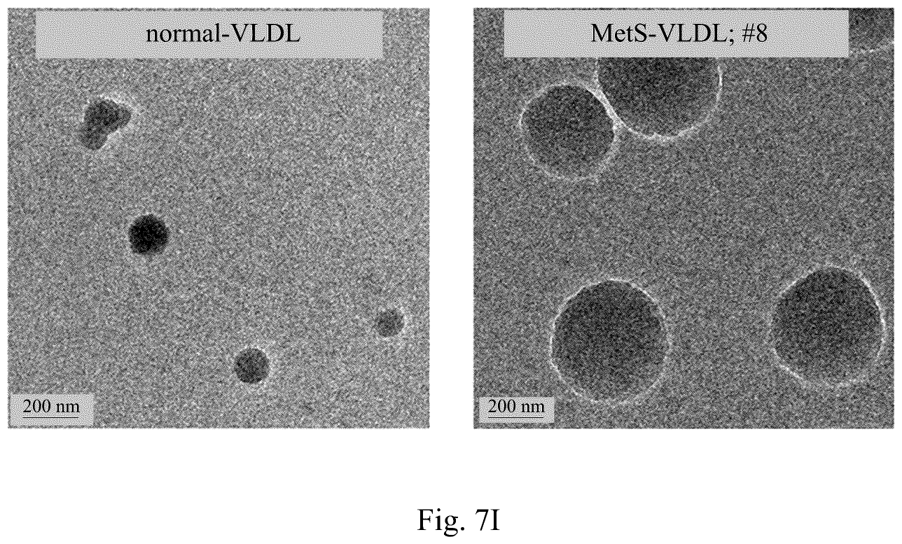

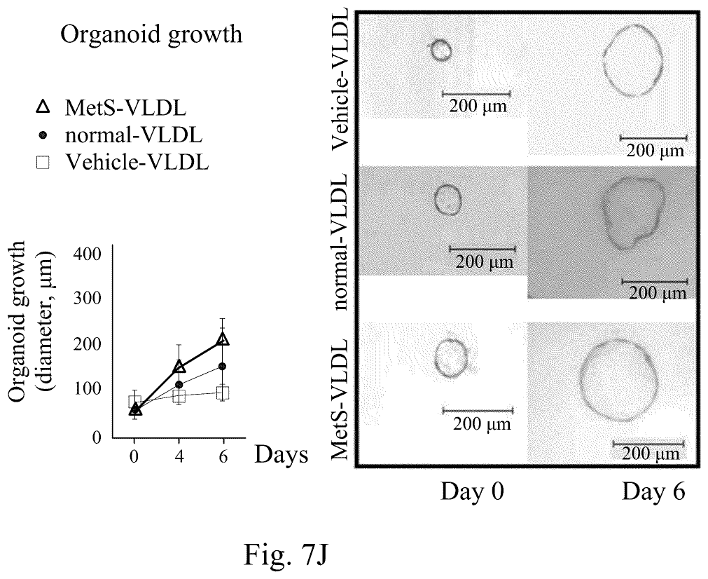

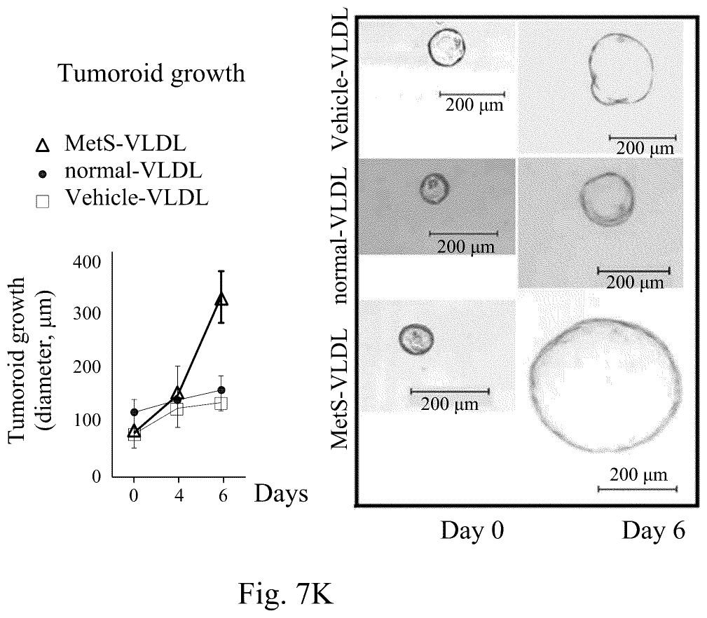

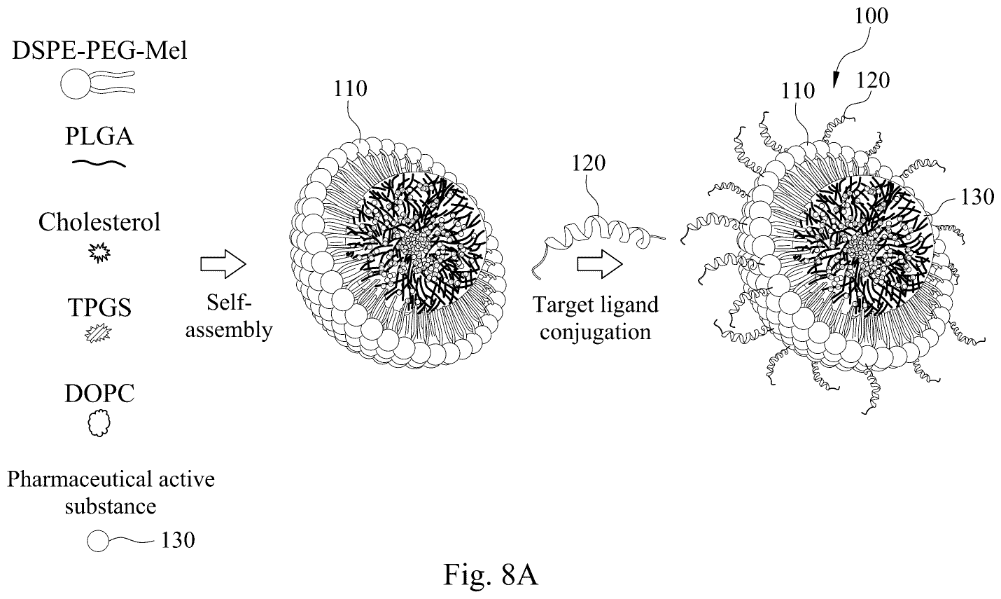

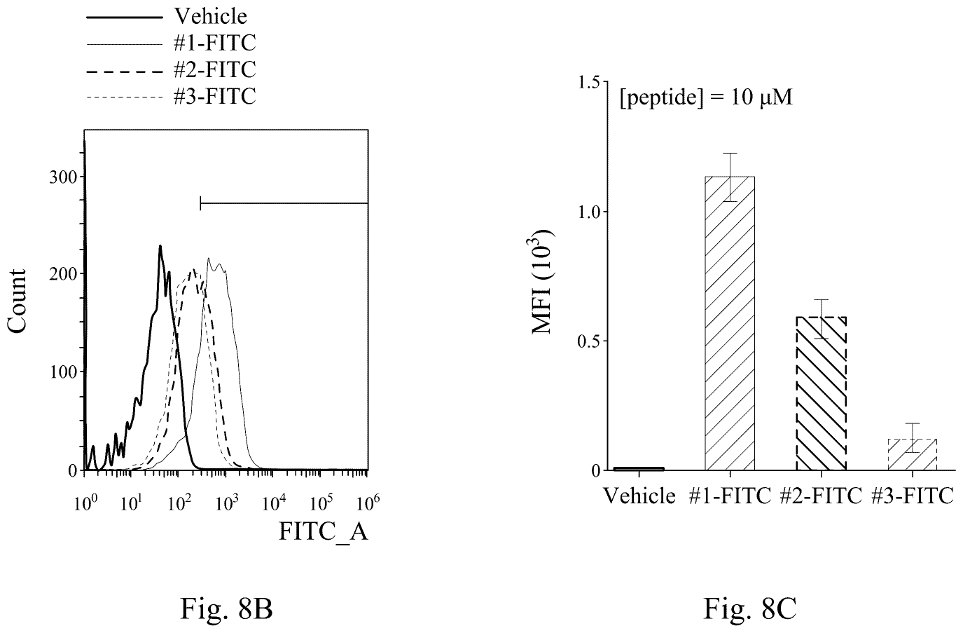

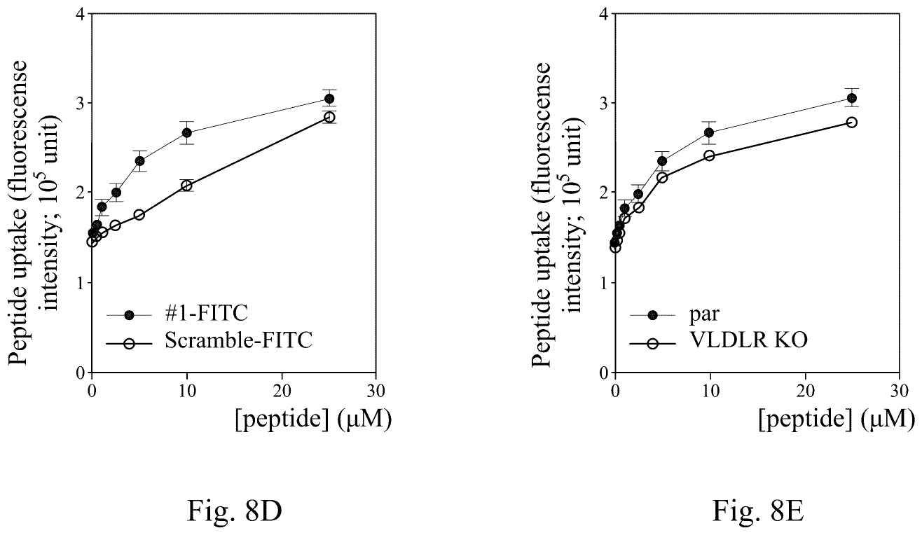

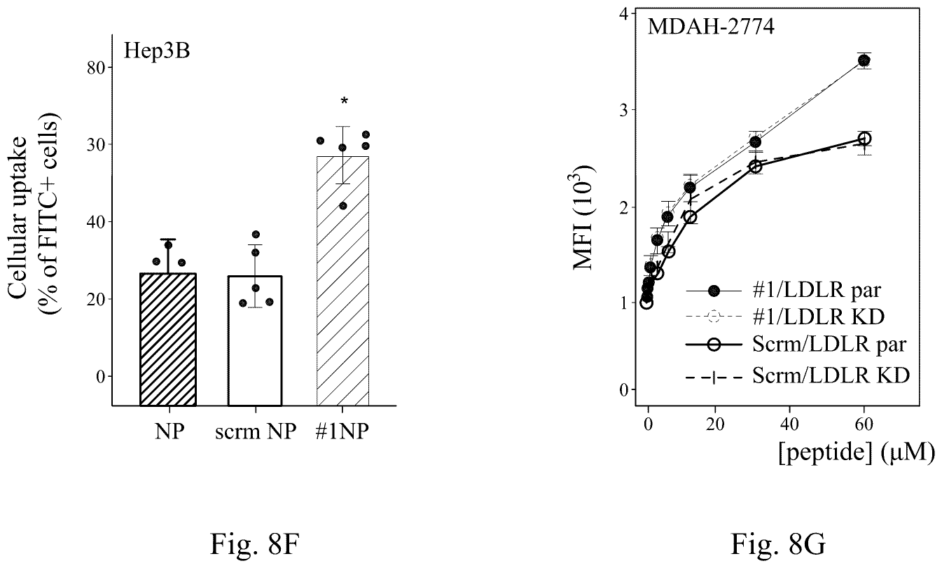

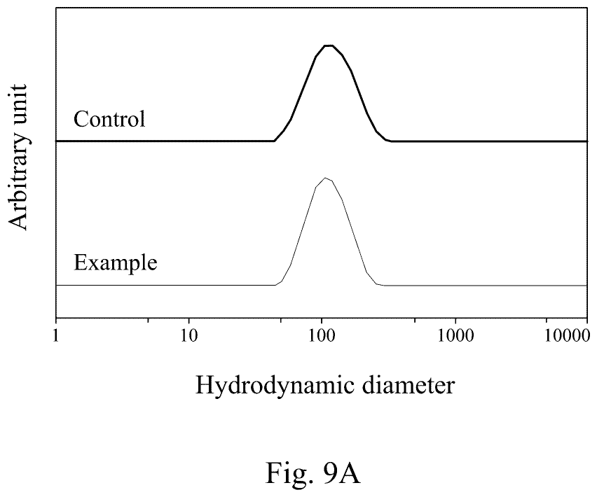

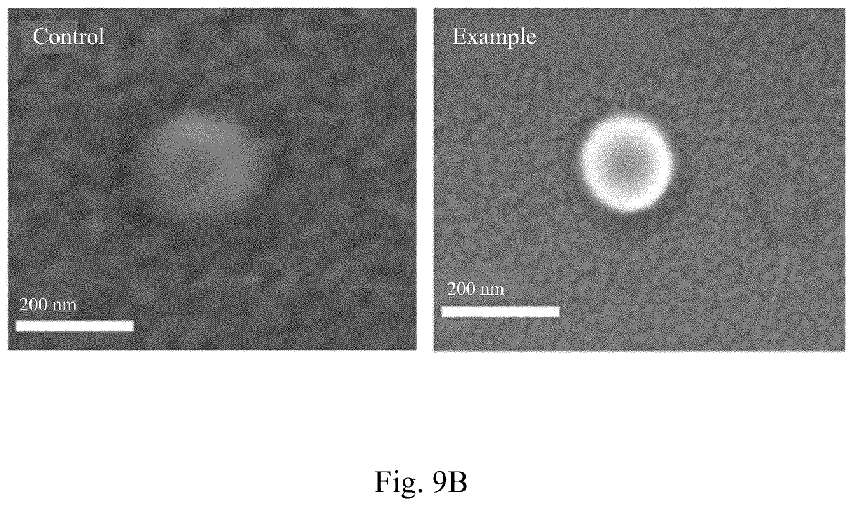

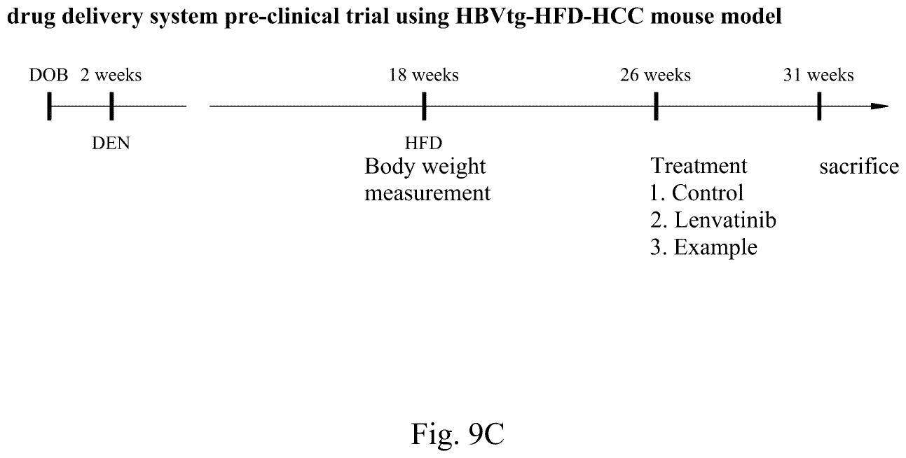

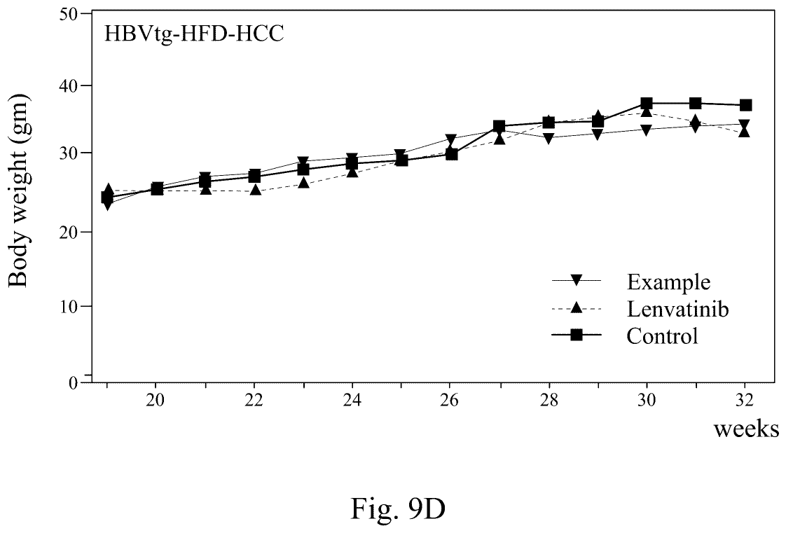

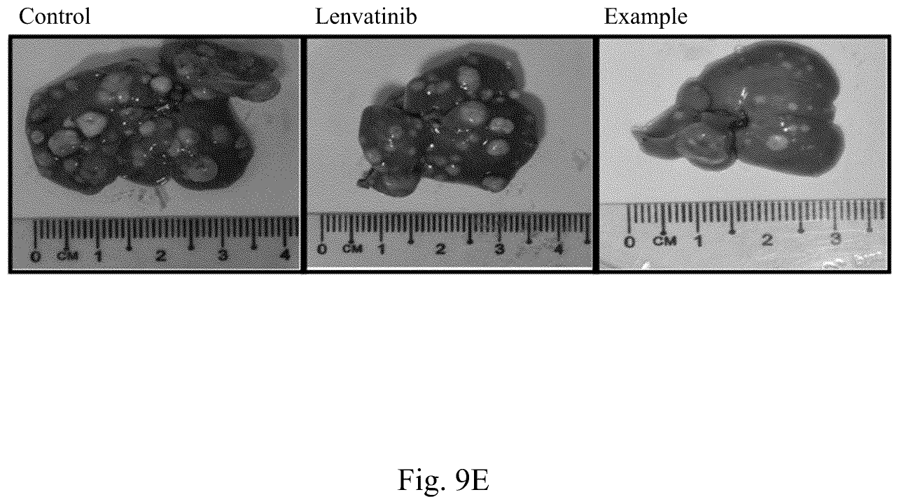

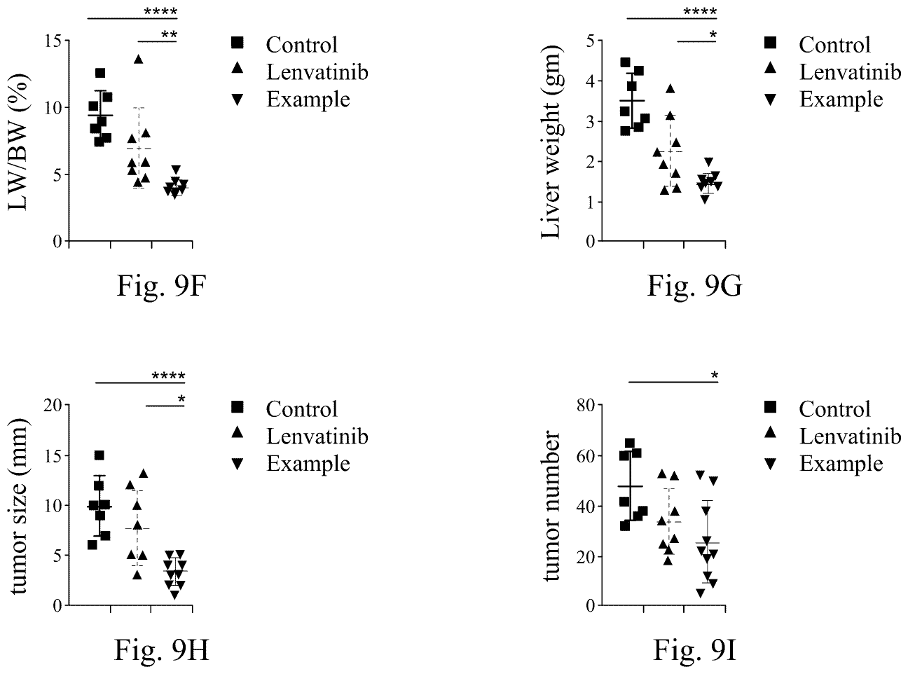

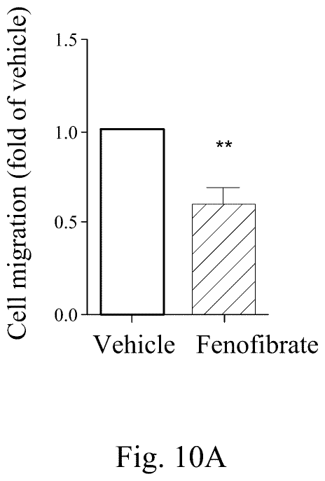

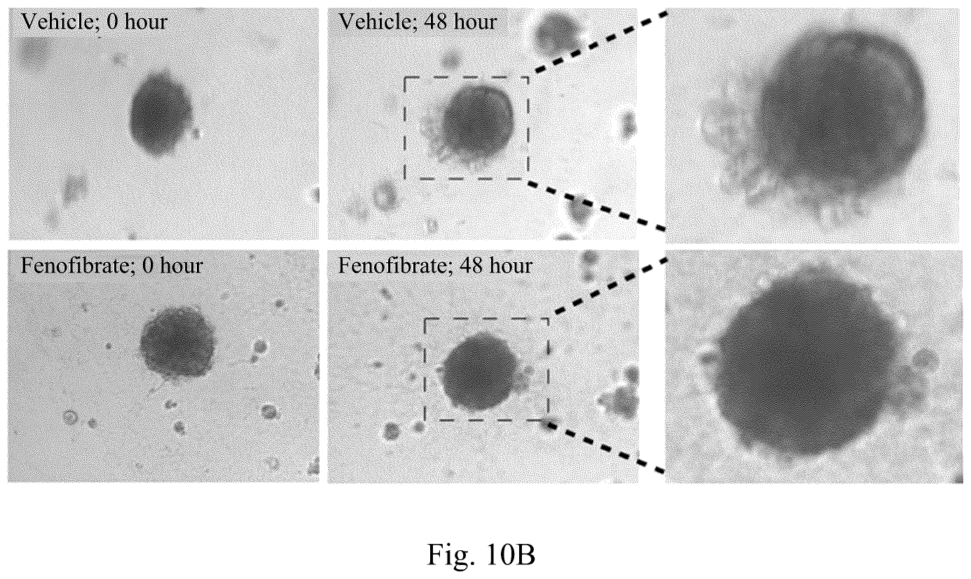

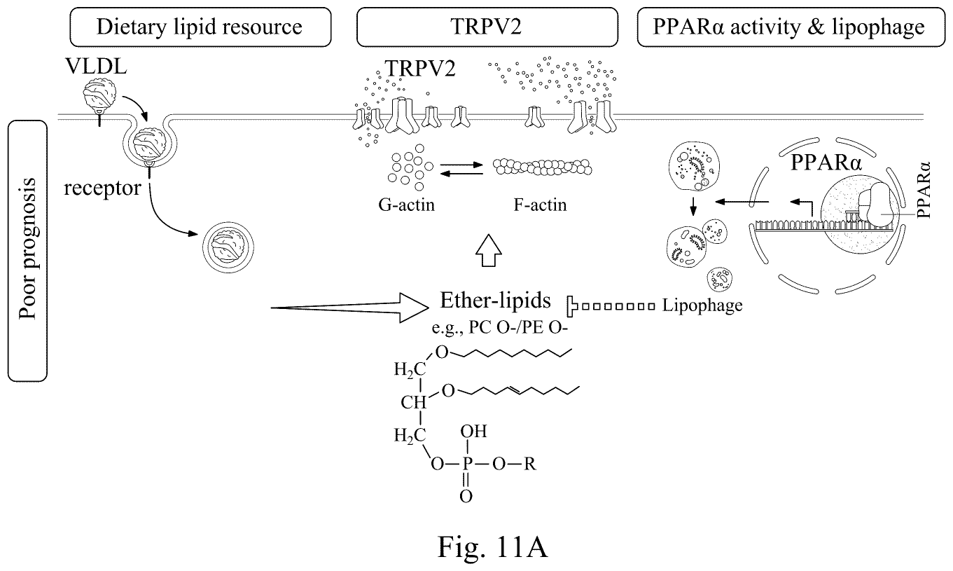

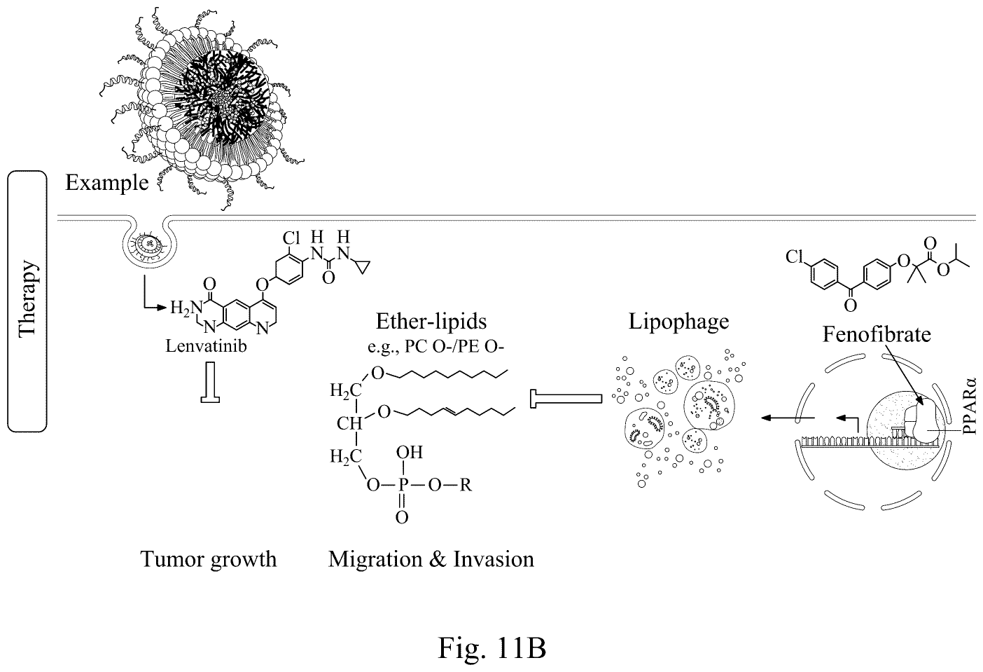

FIG. 2J shows analysis results of overall survival probability comparing the t-SNE_C group and t-SNE_D group.

FIG. 2K shows the chi-square analysis of transcriptomic grouping and lipidomic grouping.

FIG. 2L shows the KEGG pathway enrichment analysis of transcriptomic grouping of the CMUH-HCC cohort patients.

FIG. 3A shows the ranking of gene numbers that lipid classes abundance correlated with gene expressions.

FIG. 3B shows the Venn diagram of three top ranked ether-lipids genes.

FIG. 3C shows the significance ranking of the pathway enrichment analysis of 758 genes.

FIG. 3D shows the correlation between IHC staining scores of integrin-α5 and PE O— abundance.

FIG. 4A, FIG. 4B and FIG. 4C show analysis results of overall survival probability comparing high and low abundance samples of very-long-chain fatty acyl (VLCFA), odd-numbered fatty-acyl-carbon chain (Odd-FA), and polyunsaturated fatty-acyl chain (PUFA) within PC O-of the t-SNE_A group.

FIG. 4D, FIG. 4E and FIG. 4F show analysis results of overall survival probability comparing high and low abundance samples of VLCFA, Odd-FA, and PUFA within PE O— of the t-SNE_A group.

FIG. 4G, FIG. 4H and FIG. 4I show the analysis of cell migration activity, cell invasion activity, and cytoskeletal reorganization capacity in human liver cancer cell lines co-treated with VLCFA/PUFA PC O-and tranilast.

FIG. 4J shows the analysis of lipoprotein receptor mRNA expression compared tumor to normal tissue.

FIG. 5A shows analysis results of the protein expression of VLDLR in specimens from the CMUH-HCC cohort.

FIG. 5B shows analysis results of the classification of VLDLR expression into VLDLR+specimens and VLDLR-specimens.

FIG. 5C shows the lipidomic analysis comparing VLDLR+/VLDLR-with t-SNE_A/t-SNE_B groups.

FIG. 5D shows analysis results of overall survival probability comparing VLDLR+ and VLDLR-specimens from the CMUH-HCC cohort.

FIG. 5E shows analysis results of overall survival probability of VLDLR+ versus VLDLR-specimens, along with those of t-SNE_A versus t-SNE_B lipidomic profiles for the specimens from the HCC cohort.

FIG. 6A, FIG. 6B, FIG. 6C, FIG. 6D and FIG. 6E show analysis results of the VLDLR expression in different CMUH-HCC cohort and its effect on HCC.

FIG. 7A shows the association of lipoprotein-related parameters with VLDLR+/VLDLR-fold changes.

FIG. 7B shows analysis results of overall survival probability comparing VLDLR+and VLDLR-specimens along with MetS+ and MetS-specimens from the CMUH-HCC cohort.

FIG. 7C shows the protein expression and the mRNA expression of VLDLR in tumor (T) and normal parental (N) livers from the HBVtg-HCC mice.

FIG. 7D shows the representative photographs of liver specimens collected from the WT HBVtg-HFD-HCC mouse or the L_vldlr-KO HBVtg-HFD-HCC mouse.

FIG. 7E, FIG. 7F, FIG. 7G and FIG. 7H show the comparison of various parameters between the WT HBVtg-HFD-HCC mouse and the L_vldlr-KO HBVtg-HFD-HCC mouse.

FIG. 7I shows the cryo-transmission electron microscopy images of normal-VLDL and MetS-VLDL.

FIG. 7J shows the organoid growth analysis after VLDL treatment.

FIG. 7K shows the tumoroid growth analysis after VLDL treatment.

FIG. 8A is a schematic diagram showing the structure and preparation of a drug delivery system according to the present disclosure.

FIG. 8B shows analysis results of the mean fluorescence intensity in Tong cells after treatment with #1-FITC, #2-FITC, and #3-FITC.

FIG. 8C shows the quantitative results corresponding to FIG. 8B.

FIG. 8D shows analysis results of the uptake of #1-FITC and scramble-FITC into Tong cells.

FIG. 8E shows analysis results of the uptake of #1-FITC in parental Tong cells and VLDLR KO Tong cells.

FIG. 8F shows analysis results of the cellular uptake efficacy of NP, scrm NP, and #1NP.

FIG. 8G shows analysis results of the uptake of #1-FITC and scramble-FITC in LDLR parental MDAH-2774 cell or LDLR knockout MDAH-2774 cell.

FIG. 9A shows the representative images of the hydrodynamic diameter of Control and Example.

FIG. 9B shows the electron microscope images of Control and Example.

FIG. 9C is a schematic of the therapeutic strategy of the drug delivery system of the present disclosure in pre-clinical trials using the HBVtg-HFD-HCC mouse model.

FIG. 9D shows body weight change of different groups of the HBVtg-HFD-HCC mice 26 weeks to 31 weeks after treatment.

FIG. 9E shows the representative images of the tumor samples of the HBVtg-HFD-HCC mice in different groups.

FIG. 9F, FIG. 9G, FIG. 9H and FIG. 9I show the comparison of various parameters in different groups of the HBVtg-HFD-HCC mice.

FIG. 10A shows the quantitative results of the 3D invasion assay.

FIG. 10B shows microscopic images of the 3D invasion assay at 0 hour and 48 hours.

FIG. 11A shows the schematic representation of the proposed mechanism by which dietary lipids promote cancer progression.

FIG. 11B shows the schematic representation of the mechanism by which the drug delivery system and/or the treatment kit of the present disclosure inhibits tumor growth.

### DETAILED DESCRIPTION

Unless defined otherwise, all scientific or technical terms used herein have the same meaning as those understood by persons of ordinary skill in the art to which the present disclosure belongs. Any method and material similar or equivalent to those described herein can be understood and used by those of ordinary skill in the art to practice the present disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims of the present disclosure are approximate and can vary depending upon the desired properties sought by the present disclosure.

The terms “treatment,” “treating,” and “treat” herein generally refer to obtaining a desired pharmacological and/or physiological effect. The effect can be preventive in terms of completely or partially preventing a disease, a disorder, or a symptom thereof, and can be therapeutic in terms of a partial or complete cure for a disease, disorder, and/or symptoms attributed thereto. The term “treatment” used herein covers any treatment of a disease in a mammal (preferably a human) and includes suppressing development of the disease, the disorder, or the symptom thereof in a subject or relieving or ameliorating the disease, the disorder, or the symptom thereof in the subject.

The terms “individual,” “subject,” and “patient” herein are used interchangeably and refer to any mammalian animals for which diagnosis, treatment, or therapy is desired. The mammalian animals include, but are not limited to, humans, non-human primates, canines, felines, murines, bovines, equines, porcines, sheeps, deers, wolfs, foxes, and rabbits.

The term “effective amount” refers to the amount of an active agent or a pharmaceutical composition that is sufficient to bring about a therapeutic effect on a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

In the present disclosure, important cancer lipid metabolites as prognostic markers, for the first time, was discovered by using high-throughput multi-omics approaches, and delineated the differential effects of mRNA and lipid expression on cancer prognosis. Among the prognostic lipidomes, ether-lipids were the most dominant. The role of ether-lipids in enhancing cell mobility was demonstrated, notably through the activation of transient receptor potential vanilloid 2 (TRPV2) and the rearrangement of actin filaments. The abundance of ether-lipids may be attributed to downregulated peroxisome proliferator-activated receptor a (PPARa) expression. PPARa increased lipophagy activity through the PPARa to lipophagy to ether-lipids to cell migration regulatory axis, which is crucial for prognosis.

In at least one embodiment of the present disclosure, a drug delivery system includes a very low density lipoprotein (VLDL) carrier, a target ligand and a pharmaceutically active ingredient. The target ligand is conjugated to the very low density lipoprotein carrier, and the target ligand has a binding specificity to a very low density lipoprotein receptor (VLDLR). The pharmaceutically active ingredient is encapsulated in the very low density lipoprotein carrier.

The term “delivery system” as used in the present disclosure, may be a small nanoparticle, such as a nanoparticle less than 200 nm in diameter, used for purification and transport for instance to cross the blood-brain barrier.

In at least one embodiment of the present disclosure, the target ligand can include a sequence of SEQ ID NO: 1, SEQ ID NO: 2 or SEQ ID NO: 3. In at least one embodiment of the present disclosure, the very low density lipoprotein carrier can be a very low density lipoprotein mimicking nanoparticle. In at least one embodiment of the present disclosure, the very low density lipoprotein carrier can be a sphere with a particle size ranging from 100 nm to 200 nm. For example, the particle size of the sphere can be 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 195 nm, 196 nm, 197 nm, 198 nm, or 199 nm, but the present disclosure is not limited thereto. In at least one embodiment of the present disclosure, a polydispersity index (PDI) of the very low density lipoprotein carrier can be 0.1 to 0.3. For example, the PDI can be 0.15, 0.16, 0.17, 0.18, 0.19, 0.20, 0.25, 0.26, 0.27, 0.28, or 0.29, but the present disclosure is not limited thereto. In at least one embodiment of the present disclosure, a zeta potential of the very low density lipoprotein carrier can be −50 mV to −10 mV. For example, the zeta potential can be −45 mV, −40 mV, −35 mV, −30 mV, −25 mV, −20 mV, or −15 mV, but the present disclosure is not limited thereto.

In at least one embodiment of the present disclosure, the pharmaceutically active ingredient can be an anticancer drug and/or an antibody. The anticancer drug can be a tyrosine kinase inhibitor. The tyrosine kinase inhibitor can be selected from the group consisting of lenvatinib (LEVIMA®), midostaurin (RYDAPT®), sorafenib (NEXAVAR®), gilteritinib (XOSPATA®), quizartinib (VANFLYTA®), pexidartinib (TURALIO®), lestaurtinib, gefitinib (IRESSA®), erlotinib (TARCEVA®), icotinib (CONMANA®), afatinib (GILOTRIF®), crizotinib (XALKORI®), osimertinib (TAGRISSO®), almonertinib (AMEILE®), alflutinib, pacritinib (VONJO®), FF-10101, CG-806, EAI045, JBJ-25-02, BLU945, BLU701, TQB3804, BBT-176, ES-072, BPI-361175, CH7233163, AG1295, AG1296, CEP-5214, CEP-7055, HM43239, MAX-40279, FYSYN, NMS-03592088, and TG02 citrate.

In at least one embodiment of the present disclosure, the anticancer drug can be selected from the group consisting of busulfan, melphalan, chlorambucil, cyclophosphamide, ifosfamide, temozolomide, bendamustine, cisplatin, mitomycin C, bleomycin, carboplatin, camptothecin, irinotecan, topotecan, doxorubicin, epirubicin, aclacinomycin, mitoxantrone, methylhydroxyellipticine, etoposide, 5-azacytidine, gemcitabine (GEMZAR®), 5-fluorouracil, methotrexate, 5-fluoro-2′-deoxyuridine, fludarabine, nelarabine, cytarabine, alanosine, pralatrexate (FOLOTYN®), pemetrexed (ALIMTA®), hydroxyurea, thioguanine, colchicine, vinblastine, vincristine, vinorelbine (NAVELBINE®), paclitaxel (TAXOL®), ixabepilone (IXEMPRA®), cabazitaxel (JEVTANA®), docetaxel (TAXOTERE®), alemtuzumab (CAMPATH®), panitumumab (VECTIBIX®), ofatumumab (ARZERRA®), bevacizumab (AVASTIN®), herceptin (HERCEPTIN®), rituximab (RITUXAN®), imatinib (GLEEVEC®), lapatinib (TYKERB®), sunitinib (SUTENT®), nilotinib (TASIGNA®), dasatinib (SPRYCEL®), pazopanib (VOTRIENT®), temsirolimus (TORISEL®), everolimus (AFINITOR®), vorinostat (ZOLINZA®), romidepsin (ISTODAX®), tamoxifen (NOLVADEX®), letrozole (FEMARA®), fulvestrant (FASLODEX®), mitoguazone, octreotide (SANDOSTATIN®), retinoic acid, arsenic trioxide (TRISENOX®), zoledronic acid (ZOMETA®), bortezomib (VELCADE®), thalidomide (THALOMID®), and lenalidomide (REVLIMID®).

In at least one embodiment of the present disclosure, the antibody can be selected from the group consisting of an anti-HER2 antibody, an anti-CD20 antibody, an anti-IL-8 antibody, an anti-VEGF antibody, an anti-CD40 antibody, an anti-CD11a antibody, an anti-CD18 antibody, an anti-lgE antibody, an anti-Apo-2 receptor antibody, an anti-tissue factor (TF) antibody; an anti-human a437 integrin antibody, an anti-EGFR antibody, an anti-CD3 antibody, an anti-CD25 antibody, an anti-CD4 antibody, an anti-CD52 antibody, an anti-Fc receptor antibody, an anti-carcinoembryonic antigen (CEA) antibody, an antibody directed against breast epithelial cells, an antibody that bind to colon carcinoma cells, an anti-CD38 antibody, an anti-CD33 antibody, an anti-CD22 antibody, an anti-EpCAM antibody, an anti-Gpllb/Illa antibody, an anti-RSV antibody, an anti-CMV antibody, an anti-HIV antibody, an anti-hepatitis antibody, an anti-CA 125 antibody, an anti-avβ3 antibody, an anti-human renal cell carcinoma antibody, an anti-human 17-1A antibody, an anti-human colorectal tumor antibody, an anti-human melanoma antibody R24 directed against GD3 ganglioside, an anti-human squamous-cell carcinoma, an anti-human leukocyte antigen (HLA) antibody, and an anti-HLA DR antibody.

In at least one embodiment of the present disclosure, a treatment kit includes the aforementioned drug delivery system and a pharmaceutically acceptable carrier.

The term “pharmaceutically acceptable” as used herein refers to an amount sufficient to produce a therapeutic effect without causing adverse side effects. Determination of such an amount can be readily made by those skilled in the art, based on commonly known medical factors, including but not limited to the type of disease, the patient's age, weight, health condition, sex, drug sensitivity, route of administration, method of administration, dosing frequency, treatment duration, and planned combination or concurrent administration of other drug(s).

For administration to mammals, the treatment kit of the present disclosure can suspend the drug delivery system in a pharmaceutically acceptable carrier. The pharmaceutically acceptable carrier can include, but is not limited to, glycerol, water, buffered saline, ethanol, and other pharmaceutically acceptable salt solutions such as phosphate and organic acid salts.

The pharmaceutically acceptable carrier can vary depending on the route of administration, including binders, lubricants, disintegrants, excipients, solubilizers, homogenizers, suspending agents, colorants, flavoring agents or a combination thereof for oral administration; buffers, preservatives, analgesics, solubilizers, isotonic agents, tranquilizers or a combination thereof for injections; and bases, excipients, lubricants, preservatives or a combination thereof for topical administration.

In at least one embodiment of the present disclosure, the treatment kit can further include a peroxisome proliferator-activated receptor a (PPARa) agonist. The PPARa agonist can be β-carotene (BCA), retinoic acid (RA) or fibrate. The fibrate can be fenofibrate or gemfibrozil.

In at least one embodiment of the present disclosure, the treatment kit can further include a transient receptor potential vanilloid 2 (TRPV2) inhibitor. The TRPV2 inhibitor can be tranilast.

In at least one embodiment of the present disclosure, a method for inhibiting a proliferation or a metastasis of a cancer cell including administering the aforementioned treatment kit to a subject in need for a treatment of a cancer. The cancer can be a very low density lipoprotein receptor expressing cancer. The very low density lipoprotein receptor expressing cancer can be a liver cancer, a breast cancer, a stomach cancer or a lung cancer. Preferably, the liver cancer can be a hepatocellular carcinoma (HCC). In at least one embodiment of the present disclosure, the treatment kit can further include a PPARa agonist. In at least one embodiment of the present disclosure, the treatment kit can further include a TRPV2 inhibitor.

In at least one embodiment of the present disclosure, transcriptomic and lipidomic profiles independently influence prognosis. Explaining ether-lipid abundance from only metabolic gene expression is difficult. In at least one embodiment of the present disclosure, it was determined that dietary ether-lipids (e.g., very-long-chain fatty acyl, odd-numbered fatty-acyl-carbon chain, and polyunsaturated fatty-acyl chain) can promote prognosis. Furthermore, such dietary lipids can be imported through the diet-related VLDL/VLDLR pathway. In the physiological state, hepatocytes exhibit extremely low VLDLR expression. In at least one embodiment of the present disclosure, VLDLR expression upregulation in nearly half of patients was observed. This pathological VLDLR upregulation led to VLDL uptake from neighboring hepatocytes. In summary, a compromised PPARα-lipophagy pathway and increased VLDL/VLDLR lipid importation may contribute to ether-lipid accumulation, which facilitates TRPV2 activation and actin filament rearrangement, thereby promoting migration, invasion, and metastasis. The Cancer Genome Atlas (TCGA) was used to analyze associations of reported genes with prognosis; a similar trend in gastric, ovarian, bladder, and breast cancers was discovered.

In at least one embodiment of the present disclosure, ether-lipids may enhance cell mobility through calcium signaling. TRPV2 expression was consistent across various analyses, both at RNA and protein levels, with or without ether-lipid treatment. In at least one embodiment of the present disclosure, tranilast could block dietary-ether-lipid induced mobility. In at least one embodiment of the present disclosure, it suggests that TRPV2 activation may be related to membrane fluidity rather than gene expression changes. Dietary ether-lipids exhibited a notable effect on cell mobility, implying a shared mechanism with endogenous ether-lipids in cancer cells. Although the precise ether-lipid effector remains unidentified, calcium signaling emerges as a key area for further research.

The following specific examples are hereby used to further illustrate the present disclosure, so that those skilled in the art can fully utilize and practice the present disclosure without over-interpretation. These test examples should not be regarded as limiting the scope of the present disclosure, but are used to illustrate the materials and methods of practicing the present disclosure.

### I. Ether-Lipids Affect Cancer Prognosis

A cohort was established to consecutively enroll patients with hepatocellular carcinoma (HCC) who underwent hepatectomy at the Organ Transplantation Center of China Medical University Hospital (CMUH), Taiwan, between 2011 and 2013, and informed consent was obtained from 110 patients. Tumor specimens and 20 mL of whole blood were collected, preprocessed, aliquoted, and stored at −80° C. for subsequent protein, RNA, and lipid extraction. Some of the specimens were embedded in paraffin for immunohistochemical (IHC) or histological examination. The CMUH-HCC cohort's data were monitored for up to 2000 days.

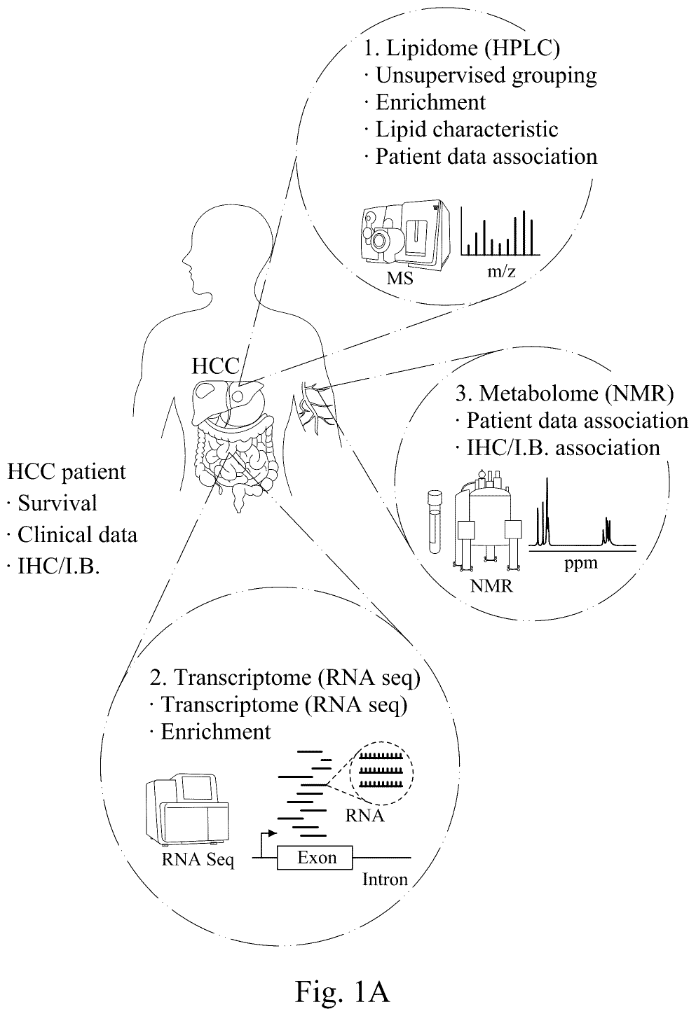

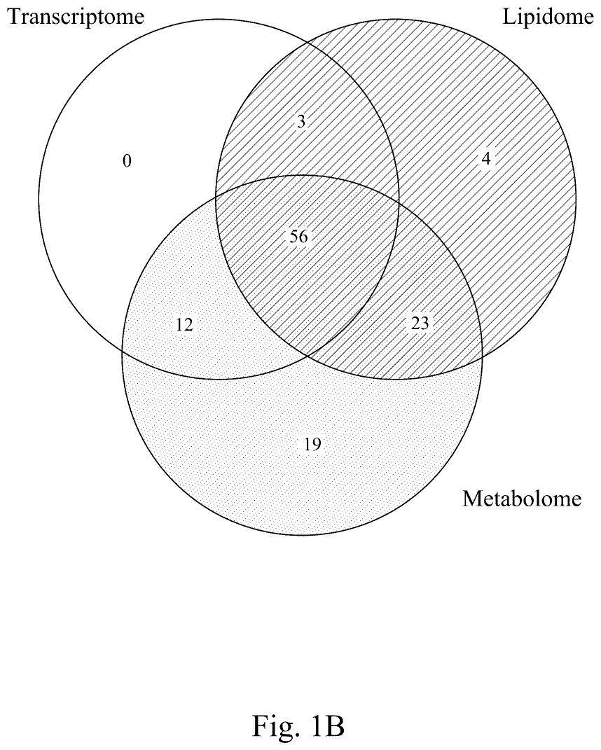

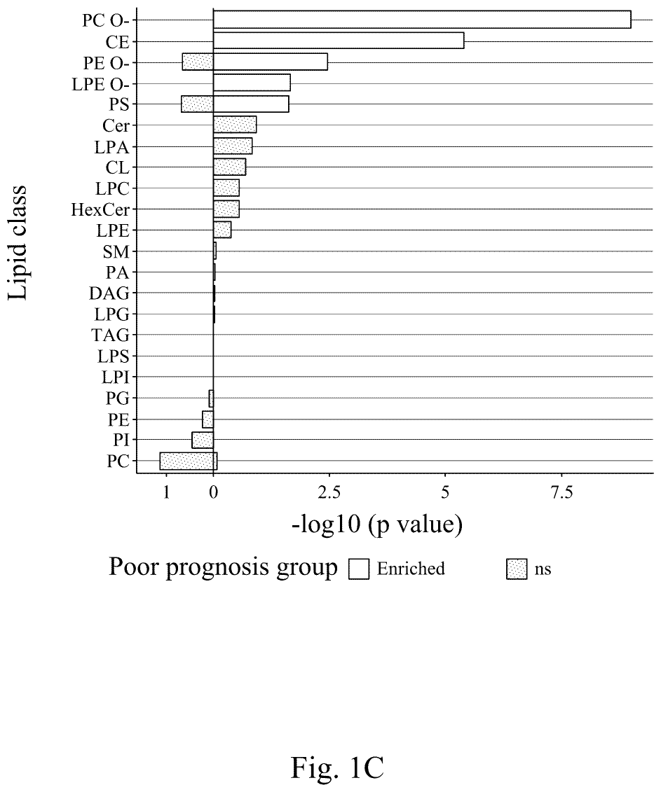

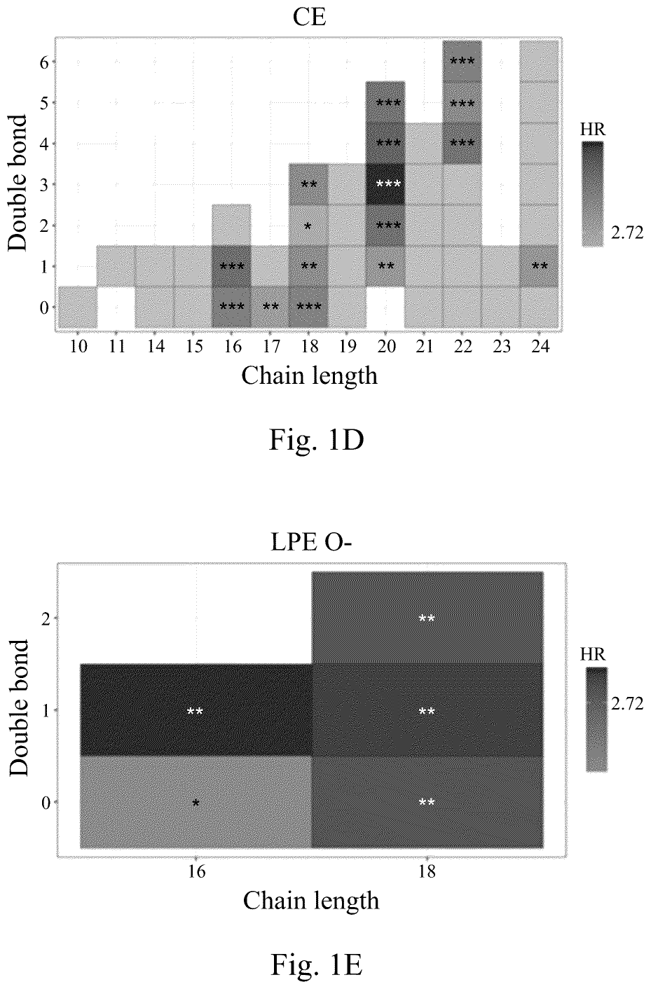

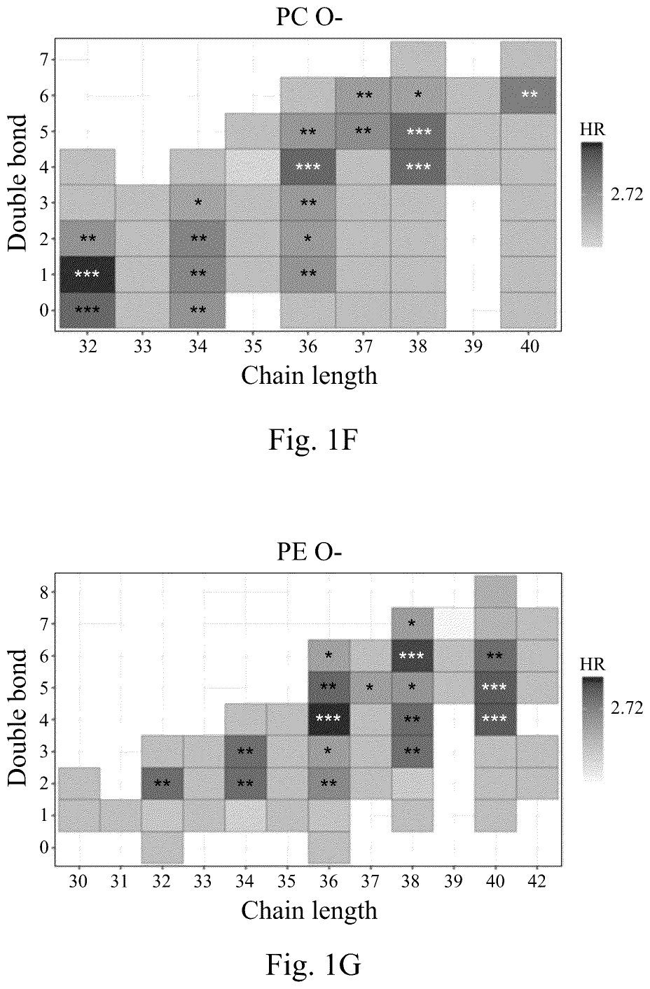

Reference is made to FIG. 1A, which is a schematic diagram showing that patients from the CMUH-HCC cohort were subjected to the lipidomic analysis, transcriptomic analysis, and metabolomic analysis. In the experiment, patients first selected from the CMUH-HCC cohort to perform lipidomic analysis and transcriptomic analysis, and the derived data were linked to metabolomic analysis data to determine the association of the tumoral lipidome, transcriptome, and metabolome with clinical features, including prognosis. Lipidomic analysis includes lipid analysis using mass spectrometry. For survival analysis, lipidomic data and clinical records, encompassing demographics, liver function parameters, and tumor characteristics, were transformed into binary variables. To investigate survival-related clinical variables, lipid species and lipid characteristics, a univariate Cox proportional hazards (Cox-PH) model was constructed. This model allowed us to calculate Log-Rank p value, hazard ratio (HR), and their corresponding 95% confidence interval (CI). Significant survival-relevant factors were identified based on a Log-Rank p value <0.05. Additionally, enrichment analysis was conducted for lipid species in relation to patient prognosis. Instead of using log2 fold change, the HR was employed to determine whether specific lipid species were beneficial or detrimental to patient survival.

For metabolomic analysis, metabolite quantification was performed on serum samples using the Nightingale high-throughput metabolomic platform based on NMR spectroscopy. The platform offers simultaneous quantification of 151 metabolic measures including routine lipids, lipoprotein subclass profiling with lipid concentrations within 14 subclasses, fatty acid composition, and various low-molecular weight metabolites, such as amino acids, ketone bodies, and gluconeogenesis-related metabolites, in molar concentration units. Additionally, 18 composite indices derived from the 151 metabolic measures were calculated. The average success rate of metabolite quantification was 99%.

In addition, regarding data exclusion, imputation, and transformation, when conducting the analysis of lipidomic dataset and metabolomic dataset, a data filtering process was applied to exclude lipid species, lipid characteristics, or metabolites with a missing value rate exceeding 70% to reduce the noise. For the remaining missing values or values below the limit of detection, they were imputed using half of the lowest observed level of the corresponding lipids or metabolites. To address the skewed distribution of expression measurements, a log transformation was further performed before conducting differential lipid expression analyses. Additionally, for survival analysis, the median value of each lipid or metabolite was calculated and used to stratify patients into two groups.

Reference is made to FIG. 1B and Table 1. FIG. 1B is a Venn diagram plot for analysis of the CMUH-HCC cohort for lipidomic analysis, transcriptomic analysis, and metabolomic analysis, showing the number of patients in each group of samples. Table 1 shows demography of the CMUH-HCC cohort patients, which shows clinical features association to risks of death (overall survival time; hazard ratio, HR). In Table 1, TNM represents tumor, node, and metastasis, and METAVIR represents meta-analysis of histological data in viral hepatitis.

TABLE 1
Demography of the CMUH-HCC cohort patients

| Variable | HR (95% CI) | p value |
|---|---|---|
| TNM stage, III-IV (%) | 5.11 (2.43-10.78) | <0.001 |
| Metastasis (%) | 3.97 (1.70-9.28) | 0.001 |
| AST (units/L), ≥40 | 3.43 (0.81-14.42) | 0.093 |
| Satellite nodule (%) | 3.03 (1.34-6.83) | 0.008 |
| Sex, male (%) | 2.86 (0.87-9.42) | 0.085 |
| Tumor size (cm), ≥5 | 2.72 (1.31-5.66) | 0.007 |
| AFP (ng/ml), ≥400 (%) | 2.67 (1.26-5.65) | 0.011 |
| Recurrence | 2.55 (1.25-5.23) | 0.010 |
| Vascular invasion | 2.48 (1.21-5.10) | 0.013 |
| Metabolic syndrome | 2.46 (1.14-5.32) | 0.022 |
| Child Pugh score, B-C | 2.30 (1.09-4.87) | 0.029 |
| Diabetes mellitus | 2.18 (1.06-4.46) | 0.033 |
| Albumin (g/dL), ≤3.4 | 1.97 (0.95-4.09) | 0.068 |
| Bilirubin (mg/dl), ≥1.2 | 1.96 (0.94-4.07) | 0.072 |
| Lymph node metastasis | 1.68 (0.75-3.78) | 0.208 |
| BMI, ≥25 | 1.63 (0.78-3.38) | 0.192 |
| Creatinine (mg/dL), ≥1.3 | 1.62 (0.70-3.78) | 0.262 |
| Hypertension | 1.62 (0.79-3.34) | 0.190 |
| ALT (units/L), ≥40 | 1.44 (0.62-3.35) | 0.402 |
| Smoking | 1.42 (0.68-2.99) | 0.351 |
| Ascites | 1.09 (0.49-2.46) | 0.830 |
| Cirrhosis, METAVIR stage F4 | 1.07 (0.51-2.26) | 0.861 |
| HBsAg, positive | 1.06 (0.52-2.17) | 0.878 |
| Age (years), ≥60 | 1.04 (0.51-2.13) | 0.911 |
| Steatosis | 0.75 (0.33-1.71) | 0.490 |
| Capsule | 0.74 (0.35-1.59) | 0.446 |
| Platelet count (10⁹/L), ≤150 | 0.54 (0.26-1.11) | 0.095 |
| HCVab, positive | 0.38 (0.15-0.95) | 0.039 |

Reference is made to Table 2 and FIG. 1C to FIG. 1G. Table 2 shows ranking of association levels of lipid classes to HR in the CMUH-HCC cohort patients. FIG. 1C shows the ranking of association levels of lipid classes to HR in the CMUH-HCC cohort patients. FIG. 1D shows the double bond number and carbon length of acyl chain of cholesterol-ester (CE) association to HR in the CMUH-HCC cohort patients. FIG. 1E shows the Double bond number and carbon length of acyl chain of ether-linked lyso-phosphatidyl-ethanolamine (LPE O—) association to HR in the CMUH-HCC cohort patients. FIG. 1F shows the double bond number and carbon length of acyl chain of ether-linked phosphatidyl-choline (PC O—) association to HR in the CMUH-HCC cohort patients. FIG. 1G shows the double bond number and carbon length of acyl chain of ether-linked phosphatidyl-ethanolamine (PE O—) association to HR in the CMUH-HCC cohort patients.

TABLE 2
Ranking of association levels of lipid classes
to HR in the CMUH-HCC cohort patients

| Lipid class | HR (95% CI) | p value |
|---|---|---|
| Cholesterol-ester (CE) | 6.89 (2.36-20.13) | <0.001 |
| Lyso-phosphatidic acid (LPA) | 5.57 (2.08-14.9) | <0.001 |
| Ceramide (Cer) | 5.22 (1.96-13.95) | <0.001 |
| Lyso-phosphatidyl-choline (LPC) | 5.17 (1.94-13.81) | <0.001 |
| Eether-linked | 4.20 (1.67-10.54) | 0.001 |
| lyso-phosphatidyl- | | |
| ethanolamine (LPE O—) | | |
| Ether-linked phosphatidyl- | 4.05 (1.61-10.16) | 0.001 |
| choline (PC O—) | | |
| Hexosyl-ceramide (HexCer) | 3.20 (1.33-7.67) | 0.006 |
| Sphingomyeline (SM) | 3.14 (1.31-7.53) | 0.007 |
| Phosphatidyl-serine (PS) | 3.08 (1.29-7.39) | 0.008 |
| Ether-linked phosphatidyl- | 2.53 (1.09-5.86) | 0.025 |
| ethanolamine (PE O—) | | |
| Lyso-phosphatidyl-serine (LPS) | 2.36 (1.02-5.47) | 0.039 |
| Diacylglycerol (DAG) | 2.04 (0.09-4.62) | 0.081 |
| Phosphatidic acid (PA) | 2.01 (0.89-4.56) | 0.087 |
| Ether-linked lyso-phosphatidyl- | 1.98 (0.90-4.34) | 0.082 |
| choline (LPC O—) | | |
| Phosphatidyl-choline (PC) | 1.93 (0.85-4.36) | 0.109 |
| Cardiolipin (CL) | 1.89 (0.83-4.28) | 0.121 |
| Lyso-phosphatidyl-glycerol (LPG) | 1.85 (0.82-4.20) | 0.133 |
| Phosphatidyl-inositol (PI) | 1.64 (0.74-3.65) | 0.223 |
| Lyso-phosphatidyl- | 1.61 (0.72-3.59) | 0.239 |
| ethanolamine (LPE) | | |
| Triacylglycerol (TAG) | 1.27 (0.57-2.79) | 0.557 |
| Phosphatidyl-glycerol (PG) | 1.08 (0.49-2.36) | 0.855 |
| Lyso-phosphatidyl-inositol (LPI) | 0.78 (0.35-1.71) | 0.527 |
| Phosphatidyl-ethanolamine (PE) | 0.49 (0.22-1.12) | 0.085 |

The results in Table 2 and FIG. 1C to FIG. 1G indicate that some lipids can be prognostic biomarkers. Rtsne package was further employed to visualize the samples by reducing the dimensionality of the processed lipidomic dataset. The tSNE (t-Distributed Stochastic Neighbor Embedding) algorithm was used with specific hyperparameters: output dimensionality=2, perplexity=15, and iterations=3000. Subsequently, K-means clustering was applied to segregate and label two groups of patients based on the transformed tSNE data, wherein K-means clustering is an unsupervised learning method. To ensure a robust grouping outcome, this process was repeated fifty times and calculated the average to assign each patient to their respective group. This approach allowed us to obtain a more stable and reliable grouping result. The information of the lipid reaction network is retrieved from Lipid Maps (https://www.lipidmaps.org/). The network was subsequently constructed using the R package, visNetwork, a robust tool that enables visualization and customization of networks. Within the constructed network, the representation of lipid classes and species was specifically focused on those identified as significant from lipid enrichment analysis. Accordingly, only the lipid classes and corresponding significant lipid species that emerged from the enrichment analysis were incorporated into the network, allowing for a targeted exploration of lipid interactions and behaviors in the context of specific objectives of the present disclosure.

Reference is made to FIG. 2A to FIG. 2D and Table 3. FIG. 2A shows that the lipidomic dataset of the CMUH-HCC cohort were divided into two groups by using unsupervised lipidomic cluster analysis executed through the t-SNE dimension reduction analysis. FIG. 2B shows analysis results of overall survival probability comparing the t-SNE_A group and t-SNE_B group. FIG. 2C shows the differential lipid analysis comparing t-SNE_A patients to t-SNE_B patients. FIG. 2D shows the comparison of t-SNE_A and t-SNE_B groups according to the results of enrichment analyses conducted on the identified lipid class. Table 3 shows lipidome patient demography of the CMUH-HCC cohort.

TABLE 3
Lipidome patient demography of the CMUH-HCC cohort

| | t-SNE_B | t-SNE_A | p |
|---|---|---|---|
| Variable | (n = 52) | (n = 32) | value |
| TNM stage, III-IV (%) | 9 (17.6) | 15 (51.7) | 0.002** |
| Recurrence (%) | 12 (23.1) | 15 (46.9) | 0.031* |
| Tumor size (cm) | 4.82 ± 3.46 | 7.03 ± 6.57 | 0.047* |
| Platelet count (10⁹/L) | 149.52 ± 81.55 | 190.41 ± 126.62 | 0.077 |
| HCVab, positive (%) | 21 (50) | 7 (28) | 0.124 |
| Diabetes mellitus (%) | 14 (26.9) | 14 (43.8) | 0.153 |
| Vascular invasion (%) | 17 (32.7) | 16 (50) | 0.167 |
| Capsule (%) | 40 (76.9) | 21 (65.6) | 0.317 |
| Albumin (g/dL) | 3.69 ± 0.63 | 3.55 ± 0.7 | 0.331 |
| Bilirubin (mg/dL) | 1.39 ± 1.14 | 1.72 ± 2.04 | 0.342 |
| AFP (ng/ml), | 12 (23.1) | 10 (33.3) | 0.438 |
| ≥400 (%) | | | |
| AST (units/L) | 140.19 ± 433.12 | 99.87 ± 83.44 | 0.616 |
| Steatosis (%) | 20 (39.2) | 10 (32.3) | 0.638 |
| ALT (units/L) | 81.73 ± 61.24 | 76.72 ± 55.85 | 0.708 |
| Metastasis (%) | 5 (9.6) | 4 (12.5) | 0.726 |
| Satellite nodule (%) | 6 (11.5) | 5 (15.6) | 0.741 |
| BMI | 24.25 ± 3.24 | 24.01 ± 3.66 | 0.764 |
| Lymph node | 10 (19.2) | 5 (15.6) | 0.775 |
| metastasis (%) | | | |
| Sex, male (%) | 41 (78.8) | 24 (75) | 0.790 |
| Creatinine (mg/dL) | 3.9 ± 18 | 5.09 ± 22.45 | 0.790 |
| Child Pugh | 12 (24.5) | 9 (29) | 0.795 |
| score, B-C (%) | | | |
| Cirrhosis, | 18 (36.7) | 12 (41.4) | 0.810 |
| METAVIR | | | |
| stage F4 (%) | | | |
| HBsAg, positive (%) | 27 (55.1) | 15 (50) | 0.817 |
| Age (years) | 60.37 ± 10.8 | 59.94 ± 11.43 | 0.864 |
| Smoking (%) | 16 (30.8) | 10 (31.2) | 1 |
| Hypertension (%) | 17 (32.7) | 11 (34.4) | 1 |
| Metabolic | 12 (25.5) | 6 (23.1) | 1 |
| syndrome (%) | | | |
| Ascites (%) | 14 (26.9) | 8 (25) | 1 |

In FIG. 2A, patients from the lipidomic dataset of the CMUH-HCC cohort were divided into two groups by using unsupervised lipidomic cluster analysis executed through the t-SNE dimension reduction analysis delineated: t-SNE_A group and t-SNE_B group. As shown in FIG. 2B, the t-SNE_A group exhibited a significantly poorer prognosis than did the t-SNE_B group (hazard ratio=3.84; p=0.0005). In FIG. 2C, the squares indicate lipid class, and the circles indicate lipid species. The squares of the circles with outer frame indicate down-regulation, and the squares of the circles without outer frame indicate up-regulation. The size of the square and the circle indicates lipid abundance. FIG. 3C shows the comparison of t-SNE_A and t-SNE_B groups according to the results of enrichment analyses conducted on the identified lipid class. Furthermore, as shown in Table 4, compared with the t-SNE_B group, the t-SNE_A group exhibited more aggressive clinical characteristics, such as advanced tumor-node-metastasis (TNM) stage, tumor recurrence, and larger tumors. In FIG. 2D, enrichment analyses indicated that the t-SNE_A group had significantly higher levels of ether phospholipids, cholesterol ester, phospholipids such as glycerophospholipids, and sphingolipids (including PC O—, PE O⇒, SM, Cer, ether LPE O—, and LPA), but had lower levels of neutral lipids (including TAG and DAG). These findings underscore the prognostic value of the lipidome, particularly the upregulation of ether phospholipids.

To demonstrate the cellular phenotype associated with ether-lipids, human HCC cell lines (Tong cells and Huh7 cells) were treated with PC O— and PE O—, respectively, and then wound healing assays and cell invasion assays were performed to observe whether ether-lipids would affect the cell migration activity and the cell invasion activity of the Tong cells and the Huh7 cells. Reference is made to FIG. 2E to FIG. 2H. FIG. 2E and FIG. 2F show analysis results of migration activity and invasion activity in the Tong cells and the Huh7 cells treated with PC O—, respectively. FIG. 2G and FIG. 2H show analysis results of migration activity and invasion activity in the Tong cells and the Huh7 cells treated with PE O—, respectively. The data in FIG. 2E to FIG. 2H are the averages of 3-4 independent experiments, where * indicates p<0.05, ** indicates p<0.01, and **** indicates p<0.0001. The results in FIG. 2E to FIG. 2H revealed that ether-lipids can promote cell migration and invasion of human hepatocellular carcinoma cell lines.

In addition, the transcriptomic dataset of the CMUH-HCC cohort was subjected to unsupervised classification after t-SNE dimension reduction analysis, and the overall survival probability was analyzed. Reference is made to FIG. 2I and FIG. 2J. FIG. 2I shows that the transcriptomic dataset of the CMUH-HCC cohort were divided into two groups by using unsupervised transcriptomic cluster analysis executed through the t-SNE dimension reduction analysis. FIG. 2J shows analysis results of overall survival probability comparing the t-SNE_C group and t-SNE_D group. In FIG. 2I, patients from the transcriptomic dataset of the CMUH-HCC cohort were divided into two groups by using unsupervised transcriptomic cluster analysis executed through the t-SNE dimension reduction analysis delineated: t-SNE_C group and t-SNE_D group. As shown in FIG. 2J, the t-SNE_C group exhibited a significantly poorer prognosis than did the t-SNE_D group (hazard ratio=3.69; p=0.00574), which indicated that transcriptomic profiles are potential prognostic biomarkers.

Furthermore, the transcriptomic groups (t-SNE_C and t-SNE_D) were compared with the lipidomic groups (t-SNE_A and t-SNE_B) by a chi-square analysis and Fisher's test (p=1). Reference is made to FIG. 2K, which shows the chi-square analysis of transcriptomic grouping and lipidomic grouping. The results showed that after visualizing the transcriptomic groups and the lipidomic groups through t-SNE, the chi-square analysis indicated distinct clustering therebetween.

The transcriptomic dataset was also analyzed by over-representation analysis (ORA) to explore the altered pathways. For ORA, up-regulated or down-regulated significant were collected and annotated them to the KEGG database and REACTOME database. Moreover, the topGO packages in R were adopted to calculate the topology of the GO graph. To identify significantly altered functions or pathways, gene set ORA was conducted through Fisher's exact test; the cutoff criterion was a p value of <0.05. Reference is made to FIG. 2L, which shows the KEGG pathway enrichment analysis of transcriptomic grouping of the CMUH-HCC cohort patients. As shown in FIG. 2L, the transcriptomic dataset was predominantly characterized by signals related to DNA replication and cell cycle events, which indicated a parallel prognostic significance for lipid and gene expression data.

To clarify the role of lipidome in prognostic-related gene expression, transcriptomic analysis was performed and t-SNE_A group patient and t-SNE_B group patient were compared to observe differential patterns of gene expression. Reference is made to FIG. 3A to FIG. 3D. FIG. 3A shows the ranking of gene numbers that lipid classes abundance correlated with gene expressions. FIG. 3B shows the Venn diagram of three top ranked ether-lipids genes. FIG. 3C shows the significance ranking of the pathway enrichment analysis of 758 genes. FIG. 3D shows the correlation between IHC staining scores of integrin-α5 and PE O—abundance.

The results in FIG. 3A and FIG. 3B show that the top three lipids correlating with gene expression were PC O—, LPE O—, and PE O— in the analysis of ether-lipid-associated transcriptomes, and these lipids were associated with the expression of a total of 758 genes. As shown in FIG. 3C, the top three pathways were REACTOME_extracellular matrix organization, GO_Cell adhesion, and KEGG_Focal adhesion in the significance ranking of the pathway enrichment analysis of 758 genes, and these genes indicated a predominant association with cell mobility pathways. Therefore, the correlation between the IHC staining score of HCC metastasis marker integrin-α5 and the abundance of ether lipids was further tested. The results in FIG. 3D show a positive correlation between integrin-α5 and ether-lipid abundance. Consequently, the transcriptomic profile and the lipidomic profile suggest a significant association of ether lipids with cell mobility, potentially influencing cancer prognosis.

### II. VLDL/VLDLR Lipid Importation Impairs Ether-Lipids Scavenging and Influences Prognosis

Lipid homeostasis is regulated by four mechanisms: anabolism, catabolismimportation, and exportation. The first section discusses the role of anabolism and catabolismtherefore, the second section describes the roles of importation and exportation in cancer progression.

The experiment first analyzed whether the structural characteristics of ether lipids affect the overall survival probability of t-SNE_A patients. Reference is made to FIG. 4A to FIG. 4F. FIG. 4A, FIG. 4B and FIG. 4C show analysis results of overall survival probability comparing high and low abundance samples of very-long-chain fatty acyl (VLCFA), odd-numbered fatty-acyl-carbon chain (Odd-FA), and polyunsaturated fatty-acyl chain (PUFA) within PC O— of the t-SNE_A group. FIG. 4D, FIG. 4E and FIG. 4F show analysis results of overall survival probability comparing high and low abundance samples of VLCFA, Odd-FA, and PUFA within PE O— of the t-SNE_A group. The results show that the abundance of VLCFA (C≥20) in PC O— and PE O— was positively associated with poor prognosis. Second, the abundance of Odd-FAs was positively associated with poor prognosis. Third, PUFA with a double-bond number of ≥2 were positively associated with poor prognosis. These lipid features suggest that dietary lipid sources are involved in HCC prognosis.

A previous study reported that ether-lipids enhance cell migration, possibly through TRPV2 and its downstream signaling, wherein TRPV2 is a calcium channel protein. To verify that ether lipids (such as PC O—) promote cytoskeletal reorganization mainly through the activity of TRPV2, human hepatocellular carcinoma cell lines were treated with VLCFA/PUFA PC O— [PC (O— 16: 0/20:5)] and one of the groups was co-treated with tranilast, which is a TRPV2 inhibitor. Wound healing assays and cell invasion assays were then performed, and the F-actin/G-actin ratio of cells in different groups was analyzed to observe cell migration ability, cell invasion ability and cytoskeletal reorganization capacity.

Reference is made to FIG. 4G to FIG. 4I, which show the analysis of cell migration activity, cell invasion activity, and cytoskeletal reorganization capacity in human hepatocellular carcinoma cell lines co-treated with VLCFA/PUFA PC O— and tranilast. In FIG. 4G to FIG. 4I, * indicates p<0.05, and ** indicates p<0.01. The results in FIG. 4G to FIG. 4I show that in the group co-treated with tranilast (TRPV2 inhibitor), tranilast nullified the promigratory (FIG. 4G), proinvasive (FIG. 4H), and cytoskeletal reorganization (FIG. 4I) effects of dietary PC O—. The results indicate that dietary ether-lipids promote cytoskeletal reorganization and cell mobility through TRPV2 activity.

To ascertain the role of lipid importers in HCC prognosis, gene expression in normal and tumor tissue specimens was analyzed. Reference is made to FIG. 4J, which shows the analysis of lipoprotein receptor mRNA expression compared tumor (HCC) to normal tissue. In FIG. 4J, p-adj represents adjusted p value, and *** indicates p<0.001. The results show that Stabilin 1 (STAB1), stabilin 2 (STAB2), and low-density lipoprotein receptor (LDLR) downregulation but noted low-density lipoprotein receptor-related protein 8 (LRP8) and very-low-density lipoprotein receptor (VLDLR) upregulation were observed. Thus, it was hypothesized that lipid importation through the VLDL/VLDLR pathway can play a role in cancer prognosis. The rationale for this hypothesis includes the following findings. First, dietary lipids influenced the prognostic lipidome of t-SNE_A individuals. Second, although VLDLR expression is typically low in normal liver tissues, as demonstrated by the GTEx portal, this expression is significant in cancer lesions. Third, VLDLR is a receptor for the dietary lipid transporter VLDL. Lipid import via the VLDL/VLDLR pathway may play a role in cancer prognosis. Because STAB1 and STAB2, which are responsible for LDL endocytosis and are primarily expressed in the sinusoids of the liver endothelium, were downregulated in this study, the STAB1/STAB2/LDLR pathway is less likely to be responsible for shaping prognostic lipidomic phenotypes.

To determine the association between VLDLR expression and clinical characteristics, VLDLR protein expression was detected in samples from the CMUH-HCC cohort, and HCC tumor samples (T) were compared with corresponding normal parental samples (N) to identify VLDLR+or VLDLR− samples. Reference is made to FIG. 5A and FIG. 5B. FIG. 5A shows analysis results of the protein expression of VLDLR in specimens from the CMUH-HCC cohort, and FIG. 5B shows analysis results of the classification of VLDLR expression into VLDLR+specimens and VLDLR-specimens. In FIG. 5A, VLDLR protein expression was detectable in the HCC tumor specimens. The expression of VLDLR in HCC tumor specimens was compared with that in normal parental specimens, specimens with a >2-fold increase in expression were designated as VLDLR+, and those with a <1-fold change were designated as VLDLR−. As shown in FIG. 5B, 44% of the tumor specimens exhibiting higher VLDLR expression than did the normal specimens.

Reference is made to Table 4 and FIG. 5C to FIG. 5E. Table 4 shows VLDLR expressions patient demograph of the CMUH-HCC cohort. FIG. 5C shows the lipidomic analysis comparing VLDLR+/VLDLR− with t-SNE_A/t-SNE_B groups. FIG. 5D shows analysis results of overall survival probability comparing VLDLR+ and VLDLR-specimens from the CMUH-HCC cohort. FIG. 5E shows analysis results of overall survival probability of VLDLR+ versus VLDLR− specimens, along with those of t-SNE_A versus t-SNE_B lipidomic profiles for the specimens from the HCC cohort.

In FIG. 5C, lipidomic comparisons between the VLDLR+ specimens and VLDLR-specimens revealed patterns consistent with those observed between the t-SNE_A group and t-SNE_B group. From a prognostic perspective in FIG. 5D, the VLDLR+ group exhibited poorer survival than did the VLDLR-group (HR=3.65; Log-Rank p value =0.0005). As shown in Table 4, increased VLDLR expression was associated with adverse clinical features, including vascular invasion, advanced TNM stage, and metabolic syndrome. Furthermore, survival analysis in FIG. 5E indicated synergistic effects (p=0.00014) between VLDLR expression and patient group; specifically, the HR for VLDLR expression was increased when combined with t-SNE_A effects. The results indicate that VLDLR protein expression has a synergistic effect with prognostic lipidome and can affect the prognosis and promote the progression of HCC.

TABLE 4
VLDLR expressions patient demograph of the CMUH-HCC cohort

| | VLDLR− | VLDLR+ | p |
|---|---|---|---|
| Variable | (n = 56) | (n = 44) | value |
| Tumor size (cm) | 4.25 ± 5.03 | 6.73 ± 4.05 | 0.009** |
| AFP (ng/ml), ≥400 (%) | 8 (15.1) | 17 (39.5) | 0.010** |
| Vascular invasion (%) | 14 (25) | 22 (50) | 0.012* |
| TNM stage, III-IV (%) | 10 (18.5) | 18 (42.9) | 0.013* |
| Satellite nodule (%) | 4 (7.1) | 10 (22.7) | 0.040* |
| Metabolic syndrome (%) | 8 (15.7) | 13 (35.1) | 0.044* |
| Platelet | 149.82 ± 91.41 | 186.92 ± 111.42 | 0.072 |
| count (10⁹/L) | | | |
| Creatinine (mg/dL) | 1.08 ± 0.67 | 7.4 ± 27.09 | 0.083 |
| Cirrhosis, | 26 (50) | 13 (31.7) | 0.093 |
| METAVIR | | | |
| stage F4 (%) | | | |
| Steatosis (%) | 17 (30.4) | 18 (45) | 0.197 |
| Metastasis (%) | 4 (7.1) | 7 (15.9) | 0.206 |
| BMI | 23.89 ± 3.77 | 24.83 ± 3.46 | 0.216 |
| AST (units/L) | 84.29 ± 62.46 | 161.29 ± 482.35 | 0.240 |
| Recurrence (%) | 15 (26.8) | 17 (38.6) | 0.280 |
| ALT (units/L) | 90.46 ± 101.32 | 74.36 ± 56.49 | 0.347 |
| Capsule (%) | 42 (75) | 29 (65.9) | 0.377 |
| Child Pugh | 17 (32.1) | 10 (24.4) | 0.494 |
| score, B-C (%) | | | |
| HCVab, positive (%) | 20 (47.6) | 15 (39.5) | 0.505 |
| Bilirubin (mg/dL) | 1.66 ± 1.87 | 1.44 ± 1.23 | 0.509 |
| Age (years) | 60.32 ± 9.81 | 58.95 ± 13.94 | 0.567 |
| Lymph node | 10 (17.9) | 10 (22.7) | 0.618 |
| metastasis (%) | | | |
| Smoking (%) | 16 (28.6) | 15 (34.1) | 0.664 |
| HBsAg, positive (%) | 28 (54.9) | 21 (50) | 0.680 |
| Albumin (g/dL) | 3.63 ± 0.63 | 3.59 ± 0.72 | 0.765 |
| Ascites (%) | 14 (25) | 12 (27.3) | 0.822 |
| Diabetes mellitus (%) | 20 (35.7) | 14 (31.8) | 0.832 |
| Sex, male (%) | 44 (78.6) | 34 (77.3) | 1 |
| Hypertension (%) | 19 (33.9) | 15 (34.1) | 1 |

Reference is made to FIG. 6A to FIG. 6E, which show analysis results of the VLDLR expression in different CMUH-HCC cohort and its effect on HCC. FIG. 6A to FIG. 6D show analysis results of the RNA sequencing technology-based transcriptome from TCGA dataset. FIG. 6A shows the VLDLR mRNA expression comparing normal parental and tumor of HCC. FIG. 6B shows the VLDLR+ and VLDLR− population in TCGA-HCC database is 58% and 42%, respectively. FIG. 6C shows that the patient counting of VLDLR expression fold change in tumor/normal was lay out. The biphasic pattern can be observed, which is similar with the CMUH-HCC cohort of protein expression. FIG. 6D shows that the overall survival of VLDLR+ patient and VLDLR− patient were compared, where the VLDLR+ patients showed a trend of poorer prognosis compared to VLDLR− patients (HR=1.78, Log-Rank p value=0.135). FIG. 6E shows that the VLDLR mRNA expression impacts on HCC prognosis with cDNA microarray technology-based transcriptome from KMplotter dataset. The grade 3rd and the HBV-related HCC patients were selected. The results show that the VLDLR expression positively correlated with HCC prognosis is consistent in different clinical features. Furthermore, the results of VLDLR mRNA expression were similar to those of VLDLR protein expression.

To demonstrate association between VLDL and VLDLR, the metabolomic analysis in association with VLDLR expression was conducted. Reference is made to FIG. 7A, which shows the association of lipoprotein-related parameters with VLDLR+/VLDLR− fold changes. In FIG. 7A, XXL represents chylomicrons and extremely large VLDL, XL represents very-large size, L (in the front) represents large size, M represents middle size, S represents small size, XS represents very small size, P represents particle number, L (in behind) represents total lipid, PL represents phospholipids, C represents cholesterol, CE represents cholesterol-ester, FC represents free cholesterol, and TG represents triglyceride.

In FIG. 7A, the VLDLR+/VLDLR− ratio increased as the VLDL particle size decreased. However, this trend was not observed for intermediate-density lipoprotein (IDL) particles, low-density lipoprotein (LDL) particles or high-density lipoprotein (HDL) particles. The results suggest an association between VLDL size or lipid content and VLDLR expression, suggesting the uptake of VLDL by VLDLR into tumors.

The experiment further isolated approximately 200 μg/mL of VLDL from blood samples of the CMUH-HCC cohort, and analyzed the overall survival probability of patients with metabolic syndrome (MetS) patients and non-MetS patients in relation to VLDLR expression. Reference is made to FIG. 7B, which shows analysis results of overall survival probability comparing VLDLR+ and VLDLR− specimens along with MetS+ and MetS-specimens from the CMUH-HCC cohort. In FIG. 7B, MetS promotes VLDL expression, indicating that VLDLR+ and MetS exhibited synergistic effects (p=0.0016) on HCC prognosis.

Considering the positive association of the VLDL/VLDLR pathway with cancer prognosis, it was attempted to establish a causal relationship between them by using experimental models. Accordingly, a hepatitis B virus transgenic (HBVtg) mouse model of HCC with a similar etiology to human HCC was used. Reference is made to FIG. 7C, which shows the protein expression and the mRNA expression of VLDLR in tumor (T) and normal parental (N) livers from the HBVtg-HCC mice. In the HBVtg-HCC mice, LDLR was downregulated and VLDLR was upregulated in tumor tissues compared with normal tissues (results not shown).

Moreover, to mimic human MetS, the mice were fed a high-fat diet (HFD) to establish the HBVtg-HFD-HCC mouse model. The HFD-HBVtg-HCC mouse model is suitable for studies involving VLDLR knockout (vldlr-KO). Subsequently, a tamoxifen-inducible liver-specific vldlr-KO model and produced wild-type (WT) HBVtg-HFD-HCC mice and liver-specific vldlr-KO (L_vldlr-KO) HBVtg-HFD-HCC mice were developed. The WT HBVtg-HFD-HCC mice were bred and housed in accordance with a specific protocol, and the L_vldlr-KO HBVtg-HFD-HCC mice status was confirmed using a quantitative real-time reverse transcription polymerase chain reaction assay (qRT-PCR). Notably, L_vldlr-KO did not affect the body weight of the HBVtg-HFD-HCC mice.

Reference is made to FIG. 7D to FIG. 7H. FIG. 7D shows the representative photographs of liver specimens collected from the WT HBVtg-HFD-HCC mouse or the L_vldlr-KO HBVtg-HFD-HCC mouse. FIG. 7E, FIG. 7F, FIG. 7G and FIG. 7H show the comparison of various parameters between the WT HBVtg-HFD-HCC mouse and the L_vldlr-KO HBVtg-HFD-HCC mouse, wherein the parameter in FIG. 7E is liver weight, the parameter in FIG. 7F is liver-to-body-weight ratio (LW/BW), the parameter in FIG. 7G is tumor count, and the parameter in FIG. 7G is tumor size. As shown in FIG. 7D to FIG. 7H, comparing cancer phenotypes between the WT HBVtg-HFD-HCC mice and the L_vldlr-KO HBVtg-HFD-HCC mice revealed a notable decrease in tumor visibility, and other parameters such as liver weight, liver-to-body-weight ratio, tumor count, and tumor dimensions also decreased.

To investigate whether VLDL stimulates human HCC, organoids (from normal liver tissues) or tumoroids (from tumor liver tissues) of HBV-HCC patients was cultured. Human VLDL particles from healthy subjects and MetS patients (hereinafter referred to as normal-VLDL and MetS-VLDL) were isolated and their morphology was observed by cryo-transmission electron microscopy (cryo-TEM).

Organoid and tumoroid were cultured by digesting normal liver tissue or tumor liver tissue from HBV-HCC patients to isolate single cells. The digestion solution used contained collagenase type XI (0.5 mg/mL, Sigma-Aldrich), dispase (0.2 mg/mL, Gibco) and DMEM medium (Lonza) containing 1% FBS, and then cultured at 37° C. for 30 minutes. The cells were then centrifuged at 600 rpm for 10 minutes to collect cell pellets. These cells were mixed with Matrigel (BD Bioscience), seeded on either 24- or 48-well plates, and incubated at 37° C. for 30 minutes. Once the Matrigel solidified, the medium was gently added. For oragnoid growth assay, the organoid and tumorid were treated with 10 μg/ml of normal-VLDL and MetS-VLDL for six days, and then take the images at different time points (0 day, 4 days, and 6 days) under the bright light microscope. All the images were quantified by Image J.

Reference is made to FIG. 7I to FIG. 7K. FIG. 7I shows the cryo-transmission electron microscopy images of normal-VLDL and MetS-VLDL. FIG. 7J shows the organoid growth analysis after VLDL treatment. FIG. 7K shows the tumoroid growth analysis after VLDL treatment. In FIGS. 7I, #2 and #8 represent blood samples from indicated patient numbers, and scale bar is 200 nm. In FIG. 7J and FIG. 7K, “normal-VLDL” represents organoid or tumoroid treated with normal-VLDL, “MetS-VLDL” represents organoid or tumoroid treated with MetS-VLDL, the quantitative results were the average values of at least 3 independent experiments, and the scale bar is 200 μm.

The results in FIG. 7I show that MetS-VLDL were substantially larger than normal-VLDL. In FIG. 7J, the results of the organoid growth analysis show that treatments with vehicle-VLDL, normal-VLDL, and MetS-VLDL resulted in comparable outcomes. However, the results of the tumoroid growth analysis in FIG. 7K show that the MetS-VLDL treatments significantly enhanced tumoroid growth compared with the vehicle-VLDL or normal-VLDL treatments.

The aforementioned results indicate that lipid scavenging deficits are exacerbated by VLDL/VLDLR pathway upregulation, which causes substantial ether-lipid clearance stress, resulting in poor prognosis.

### III. Drug Delivery System, Treatment Kit, and Use of the Present Disclosure

The aforementioned experimental data show the carcinogenic effect of the VLDL/VLDLR pathway. Therefore, a drug delivery system of the present disclosure includes a very low density lipoprotein (VLDL) carrier, a target ligand and a pharmaceutically active ingredient. The target ligand is conjugated to the VLDL carrier, and the target ligand has a binding specificity to a very low density lipoprotein receptor (VLDLR). The pharmaceutically active ingredient is encapsulated in the VLDL carrier. The drug delivery system of the present disclosure leverages the pathological upregulation of VLDLR in tumor cells to achieve highly specific targeting of such cells, which allows for the precise delivery of the encapsulated pharmaceutically active ingredient directly into the tumor cells.

Reference is made to FIG. 8A, which is a schematic diagram showing the structure and preparation of a drug delivery system 100 according to the present disclosure. In one embodiment, the VLDL carrier 110 can be a VLDL mimicking nanoparticle, which is self-assembled by PEGylated liposome nanoparticle (hereinafter referred to as NP), and the target ligand 120 is conjugated to the surface of the VLDL mimicking nanoparticle, and the pharmaceutically active ingredient 130 is encapsulated in the VLDL mimicking nanoparticle.

The VLDL mimicking nanoparticle was synthesized using a single-step nanoprecipitation method. In detail, 0.75 mg of poly (lactic-co-glycolic acid) (PLGA), 0.0846 mg of 1,2-distearoyl-sn-glycero-3 -phosphoethanolamine-N-[maleimide (polyethylene glycol)-2000] (DSPE-PEG(2000)-maleimide;), 0.375 mg of D-α-tocopherol polyethylene glycol 1000 succinate (TPGS), and 0.0375 mg of 1,2-Dioleoyl-sn-glycero-3-phosphocholine (DOPC) were dissolved in 40 μL of dimethyl sulfoxide (DMSO) to form an organic phase mixture. The organic phase mixture was then gradually added to 280 μL of deionized water in a dropwise manner, with continuous stirring for 30 minutes. The oil-to-water-phase volume ratio was maintained at 1/7 (v/v) for all experiments. To encapsulate the pharmaceutically active ingredient 130 in the VLDL mimicking nanoparticle, the pharmaceutically active ingredient 130 can be added to the organic phase mixture. For example, in one embodiment, the pharmaceutically active ingredient 130 to be encapsulated is lenvatinib, which is a multi-kinase inhibitor that can inhibit VEGFR1-3, FGFR1-4, RET, KIT and PDGFR, and is currently a first-line treatment for advanced liver cancer. Therefore, when preparing Example, 0.15 mg of lenvatinib was added to the above organic phase mixture and the subsequent steps are the same.

In one embodiment, the target ligand 120 can be an ApoE peptide, which includes a sequence of SEQ ID NO: 1, SEQ ID NO: 2 or SEQ ID NO: 3. First, the cellular uptake of different ApoE peptides was evaluated. The ApoE peptides used included ApoE peptide #1 having a sequence as shown in SEQ ID NO: 1 (hereinafter referred to as peptide #1), ApoE peptide #2 having a sequence as shown in SEQ ID NO: 2 (hereinafter referred to as peptide #2), and ApoE peptide #3 having a sequence as shown in SEQ ID NO: 3 (hereinafter referred to as peptide #3). A scrambled peptide with the sequence shown in SEQ ID NO: 4 was used as a control group, and each peptide was conjugated with FITC fluorescein to obtain FITC-labeled peptides (respectively #1-FITC, #2-FITC, #3-FITC and scrambled-FITC).

Tong cells were seeded in a 24-well plate at a density of 1×10⁵ cells per well. On the following day, after replacing the culture medium, 500 μL of FITC-labeled peptide solutions at concentrations of 0 mM, 0.25 mM, 0.5 mM, 1 mM, 5 mM, 10 mM, 25 mM and 50 mM were added, respectively. After 2 hours incubation, the mean fluorescence intensity of cells in each sample was measured and analyzed by flow cytometry to evaluate the peptide uptake by Tong cells.

Reference is made to FIG. 8B to FIG. 8E. FIG. 8B shows analysis results of the mean fluorescence intensity in Tong cells after treatment with #1-FITC, #2-FITC, and #3-FITC. FIG. 8C shows the quantitative results corresponding to FIG. 8B. FIG. 8D shows analysis results of the uptake of #1-FITC and scramble-FITC into Tong cells. FIG. 8E shows analysis results of the uptake of #1-FITC in parental Tong cells and VLDLR KO Tong cells, wherein par represents parental cells, and VLDLR KO represents VLDLR knockout Tong cells.

As shown in FIG. 8B and FIG. 8C, the uptake of peptide #1 by Tong cells was greater than that of peptide #2 and peptide #3. The results in FIG. 8D show that the uptake of peptide #1 by Tong cells was greater than that of scrambled peptides. In FIG. 8E, the uptake of peptide #1 by parental Tong cells was higher than that of VLDLR KO Tong cells.

Peptide #1 and scrambled peptide were each conjugated to NP to produce #1NP and scrm NP, respectively. The fluorescent dye coumarin 6 (C6) was then encapsulated into #1NP, scrm NP, and NP, with a final weight ratio of C6 to PLGA of 1:150. Hep3B cells (a human hepatocellular carcinoma cell line) were seeded into 12-well plates at a density of 5×10⁵ cells per well. After overnight incubation, C6-loaded #1NP, scrm NP, and NP were added to the cells and incubated at 37° C. for 20 minutes. The mean fluorescence intensity of the cells in each sample was then measured and analyzed using flow cytometry to assess peptide uptake by the Tong cells.

Reference is made to FIG. 8F, which shows analysis results of the cellular uptake efficacy of NP, scrm NP, and #1NP. The results show that the uptake of #1NP by Hep3B cells was greater than that of scrm NP, while the uptake of scrm NP by Hep3B cells was similar to that of NP.

In addition, LDLR parental cells or LDLR knockout MDAH-2774 cells were treated with #1-FITC and scrambled-FITC to evaluate the cellular uptake. MDAH-2774 cells are a human ovarian cancer cell line. Reference is made to FIG. 8G, which shows analysis results of the uptake of #1-FITC and scramble-FITC in LDLR parental (LDLR par) MDAH-2774 cell or LDLR knockout (LDLR KO) MDAH-2774 cell, wherein #1 represents #1-FITC, and Scrm represents scramble-FITC. As shown in FIG. 8G, the uptake rates in the parental cells and the LDLR-KO cells were similar, indicating that the peptide #1 entered the cells through VLDLR rather than LDLR. Therefore, future Example production processes should involve conjugating the peptide #1 to the VLDL mimicking nanoparticle and target VLDLR.

In the preparation of the embodiment, peptide #1 was first reduced using TCEP gel treatment, followed by incubation with the VLDL mimicking nanoparticle. Subsequently, the thiol group of peptide #1 was reacted with DSPE-PEG-Mel to form stable thioether bonds. Four hours after incubation, any residual maleimide was neutralized by introducing free cysteine, resulting in the drug delivery system of the embodiment (hereinafter referred to as the “Example”). The peptide-modified Example was collected by centrifugation at 25,000×g for 30 minutes at 25° C., and the resulting pellet was resuspended in PBS for subsequent experiments.

The prepared Example was then characterized for morphology thereof using field-emission scanning electron microscopy (ULTRA plus FESEM, Zeiss, Germany), and particle size and zeta potential thereof were analyzed using a nanoparticle size analyzer (300HS; Malvern Instruments Ltd., Worcestershire, UK). Additionally, to determine the encapsulation efficiency (E.E.) of the Example, the concentration of lenvatinib was measured using a UV-Vis spectrophotometer at wavelengths of 270 nm and 260 nm. The encapsulation efficiency was calculated using the following formula: E.E.=(amount of pharmaceutically active ingredient loaded into the VLDL carrier/total amount of pharmaceutically active ingredient added)×100%.

Reference is made to FIG. 9A, FIG. 9B and Table 5. FIG. 9A shows the representative images of the hydrodynamic diameter of Control and Example. FIG. 9B shows the electron microscope images of Control and Example. Table 5 shows characteristic analysis data of Example. Control was the VLDL mimicking nanoparticle without lenvatinib encapsulation.

TABLE 5
Characteristic analysis data of Example

| | |
|---|---|
| Particle size (nm) | 133 ± 29 |
| Polydispersity index (PDI) | 0.16 ± 0.04 |
| Zeta potential (mV) | −33 |
| Encapsulation efficiency (%) | 69 ± 5 |

The drug delivery system of the present disclosure is a type of nanoparticle, with particle size being a critical factor influencing its biodistribution, tissue penetration, and cellular uptake efficiency. The results show that the particle size of Example is 133±29 nm, which falls within the desirable size range for drug delivery systems and facilitates in vivo transport and permeability of the drug. The polydispersity index (PDI) of Example is 0.16±0.04, indicating a uniform particle size distribution. The zeta potential, an important physicochemical parameter affecting nanoparticle stability and cellular interactions in vivo, plays a key role in ensuring product consistency and stability. The zeta potential range for lipoprotein-based carriers is defined as −50 mV to −10 mV. Example exhibits a zeta potential of −33 mV, which falls within the acceptable range. In addition, the encapsulation efficiency of the Example reaches 69±5%, demonstrating satisfactory formulation quality.

To further evaluate the therapeutic efficacy of the drug delivery system of the present disclosure in vivo, a pre-clinical study was conducted using the HBVtg-HFD-HCC mouse model. Reference is made to FIG. 9C, which is a schematic of the therapeutic strategy of the drug delivery system of the present disclosure in pre-clinical trials using the HBVtg-HFD-HCC mouse model. In the experiment, HBVtg mice were intraperitoneally injected with diethylnitrosamine (DEN) at two weeks of age to induce HCC and were subsequently fed a high-fat diet (HFD) starting at 18 weeks of age. Treatment commenced at week 26 and continued through week 31. HBVtg-HFD-HCC mice were divided into three treatment groups: the first group received biweekly intravenous injections of Control; the second group received biweekly oral administration of lenvatinib (9 mg/kg); and the third group received biweekly oral administration of Example containing lenvatinib (9 mg/kg). All HBVtg-HFD-HCC mice were euthanized for analysis at 32 weeks.

Reference is made to FIG. 9D to FIG. 9I. FIG. 9D shows body weight change of different groups of the HBVtg-HFD-HCC mice 26 weeks to 31 weeks after treatment. FIG. 9E shows the representative images of the tumor samples of the HBVtg-HFD-HCC mice in different groups. FIG. 9F, FIG. 9G, FIG. 9H and FIG. 9I show the comparison of various parameters in different groups of the HBVtg-HFD-HCC mice, wherein the parameter in FIG. 9F is liver-to-body-weight ratio (LW/BW), the parameter in FIG. 9G is liver weight, the parameter in FIG. 9H is tumor size, and the parameter in FIG. 9I is tumor count.

As shown in FIG. 9D, during the treatment period, the HBVtg-HFD-HCC mice in all groups maintained similar body weight profiles, indicating minimal systemic toxicity. The results in FIG. 9E to FIG. 9I show that Example exhibited a significant effect in suppressing tumor progression, as evidenced by gross observations and tumor markers such as liver-to-body-weight ratio, liver weight, and tumor size. In contrast, lenvatinib moderately suppressed tumor growth. Furthermore, lenvatinib failed to reduce tumor number substantially, whereas Example demonstrated a strong suppressive effect on tumor number. These findings indicate that while the drug delivery system of the present disclosure cannot reverse preexisting tumorigenesis but can inhibit tumor progression.

The results of the first section of the experiment show that ether-lipids are significantly associated with tumor cell migration and may affect cancer prognosis. The PPARa downregulation is associated with reduced ether-lipids scavenging and increased cell migration activities. Hence, the potential of pharmacologically activating PPARa to mitigate metastasis risks was explored.

HCC cell were treated with fenofibrate, a PPARa agonist, and then a three-dimensional invasion assay was performed. In detail, a cell suspension of Tong cells (0.5-1×10⁴ cells/mL) was first prepared, and 200 μL of the cell suspension was dispensed into a 96-well round bottom plate. Incubate the plate in incubator for four days to formed tumor spheroid. Four days later, place the plate on ice and remove 100 μL of medium. Then dispense 100 μL of basement membrane-like matrix (BMM) containing 10 ng/ml of EGF into each well. Transfer the plate to an incubator to make the BMM solidify. One hour later, add 100 μL/well of complete growth medium containing 30 μM of fenofibrate (3× the desired final concentration). After treatment, take the images at different time point (0 hour, 24 hours, 48 hours). All the images were quantified by using Image J.

Reference is made to FIG. 10A and FIG. 10B, FIG. 10A shows the quantitative results of the 3D invasion assay, and FIG. 10B shows microscopic images of the 3D invasion assay at 0 hour and 48 hours. Within 48 hours of treatment with fenofibrate, the results show that fenofibrate suppressed the migration of the Tong cells and reduced the formation of filopodia in three-dimensional (3D) spheroid derived from the Tong cells.

Reference is made to FIG. 11A and FIG. 11B, FIG. 11A shows the schematic representation of the proposed mechanism by which dietary lipids promote cancer progression, and FIG. 11B shows the schematic representation of the mechanism by which the drug delivery system and/or the treatment kit of the present disclosure inhibits tumor growth.

As illustrated in FIG. 11A, upregulated VDLDLR uptake lipid from dietary resource accompany with PPARa mediate diminished lipid scavenging by lipophagy, trigger the ether-lipids accumulation and further mediate TRPV2 signal to activate cell migration, and finally leading to poor prognosis. In detail, ether-lipids, such as PC O— and PE O—, promote cell mobility and cancer prognosis through the VLDL/VLDLR pathway. This phenomenon occurs because cellular ether-lipids are formed in the peroxisome and endoplasmic reticulum (ER) before being exported through ABC transporters on lipid droplets (LDs). VLDLs are produced in the rough ER, sorted to the Golgi, lipid-loaded in the smooth ER, and then exported out of the hepatocyte. Therefore, the mechanism of ether-lipid accumulation might involve several steps. First, dietary lipids are enriched in VLDLs. Second, the lipid-loaded VLDLs are then internalized through VLDLR and unloaded into lysosomes and peroxisomes. Third, the dietary lipids are converted into alcohol-lipids caused by reactive oxide species. Fourth, ether-lipids are formed through alkylglycerone phosphate synthase (AGPS). Fifth, the low expression of PPARa leads to reduced lipophagic activity, resulting in diminished transport of ether-lipids to LDs for clearance. Sixth, the accumulation of ether-lipids in the smooth ER increases the likelihood of their incorporation into VLDLs. Seventh, the ether-lipid-VLDLs are secreted into circulation. Eighth, tumor overexpression of VLDLR causes the recycling of these ether-lipid-loaded VLDLs from neighboring hepatocytes. Finally, the aforementioned steps form a pathological positive feedback loop.

As illustrated in FIG. 11B, the drug delivery system of the present disclosure employs a VLDL carrier conjugated with a target ligand that specifically binds to VLDLR, rather than LDLR, and encapsulates the pharmaceutically active ingredient within the VLDL carrier. Based on the pathological upregulation of VLDLR in tumor cells, the drug delivery system enables highly specific targeting of such cells and allows for the precise delivery of pharmaceutically active ingredient encapsulated therein, thereby inhibiting tumor growth. Furthermore, due to the hydrophobic nature of the VLDL-based carrier, hydrophobic metastasis inhibitors (such as lenvatinib or retinoic acid) can be effectively encapsulated within the VLDL carrier. The treatment kit of the present disclosure can further include a PPARα agonist (such as fenofibrate or β-carotene), which induces lipophagy, thereby enhancing the therapeutic efficacy and achieving superior inhibition of tumor metastasis.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1. A drug delivery system, comprising:
a very low density lipoprotein carrier;
a target ligand conjugated to the very low density lipoprotein carrier, wherein the target ligand has a binding specificity to a very low density lipoprotein receptor;
and
a pharmaceutically active ingredient encapsulated in the very low density lipoprotein carrier.

2. The drug delivery system of claim 1, wherein the target ligand comprises a sequence of SEQ ID NO: 1, SEQ ID NO: 2 or SEQ ID NO: 3.

3. The drug delivery system of claim 1, wherein the very low density lipoprotein carrier is a very low density lipoprotein mimicking nanoparticle.

4. The drug delivery system of claim 3, wherein the very low density lipoprotein carrier is a sphere with a particle size ranging from 100 nm to 200 nm.

5. The drug delivery system of claim 3, wherein a polydispersity index of the very low density lipoprotein carrier is 0.1 to 0.3.

6. The drug delivery system of claim 3, wherein a zeta potential of the very low density lipoprotein carrier is −50 mV to −10 mV.

7. The drug delivery system of claim 1, wherein the pharmaceutically active ingredient is an anticancer drug and/or an antibody.

8. The drug delivery system of claim 7, wherein the anticancer drug is a tyrosine kinase inhibitor.

9. The drug delivery system of claim 8, wherein the tyrosine kinase inhibitor is selected from the group consisting of lenvatinib, midostaurin, sorafenib, gilteritinib, quizartinib, pexidartinib, lestaurtinib, gefitinib, erlotinib, icotinib, afatinib, crizotinib, osimertinib, almonertinib, alflutinib, pacritinib, FF-10101, CG-806, EAI045, JBJ-25-02, BLU945, BLU701, TQB3804, BBT-176, ES-072, BPI-361175, CH7233163, AG1295, AG1296, CEP-5214, CEP-7055, HM43239, MAX-40279, FYSYN, NMS-03592088, and TG02 citrate.

10. A treatment kit, comprising:
the drug delivery system of claim 1; and
a pharmaceutically acceptable carrier.

11. The treatment kit of claim 10, further comprising a peroxisome proliferator-activated receptor a (PPARα) agonist.

12. The treatment kit of claim 11, wherein the PPARα agonist is β-carotene (βCA), retinoic acid (RA) or fibrate.

13. The treatment kit of claim 12, wherein the fibrate is fenofibrate or gemfibrozil.

14. The treatment kit of claim 10, further comprising a transient receptor potential vanilloid 2 (TRPV2) inhibitor.

15. The treatment kit of claim 14, wherein the TRPV2 inhibitor is tranilast.

16. A method for inhibiting a proliferation or a metastasis of a cancer cell comprising administering the treatment kit of claim 10 to a subject in need for a treatment of a cancer.

17. The method of claim 16, wherein the cancer is a very low density lipoprotein receptor expressing cancer.

18. The method of claim 17, wherein the very low density lipoprotein receptor expressing cancer is a liver cancer, a breast cancer, a stomach cancer or a lung cancer.

19. The method of claim 16, wherein the treatment kit further comprises a peroxisome proliferator-activated receptor a (PPARα) agonist.

20. The method of claim 16, wherein the treatment kit further comprises a transient receptor potential vanilloid 2 (TRPV2) inhibitor.


SEQUENCE LISTING (4 sequences)

SEQ ID NO: 1 - protein, 20 aa, synthetic construct
EELRVRLASHLRKLRKRLLR

SEQ ID NO: 2 - protein, 20 aa, synthetic construct
RLASHLRKLRKRLLRDADDL

SEQ ID NO: 3 - protein, 20 aa, synthetic construct
LLRVLRKREERRLLLRKASH

SEQ ID NO: 4 - protein, 20 aa, synthetic construct
LLRVLRKREERRLLLRKASH